(19) United States
(12) Patent Application Publication
(10) Pub. No.: US 2022/0041583 A1
(43) Pub. Date: Feb. 10, 2022

(54) IMMUNOMODULATORS, COMPOSITIONS AND METHODS THERE OF

(71) Applicant: BETTA PHARMACEUTICALS CO., LTD, Zhejiang (CN)

(72) Inventors: Yiqian WANG, Daxing, Beijing (CN); Yao ZHANG, Daxing, Beijing (CN); Bang FU, Daxing, Beijing (CN); Jiabing WANG, Daxing, Beijing (CN)

(73) Assignee: BETTA PHARMACEUTICALS CO., LTD, Zhejiang (CN)

(21) Appl. No.: 17/275,450

(22) PCT Filed: Sep. 12, 2019
(86) PCT No.: PCT/CN2019/105685

§ 371 Date: Mar. 11, 2021

(30) Foreign Application Priority Data
Sep. 13, 2018 (CN) PCTCN2018105582

(51) Int. Cl. C07D 413/14; C07D 413/06; C07D 263/57; C07D 498/04

(52) U.S. Cl. CPC C07D 413/14; C07D 498/04; C07D 263/57; C07D 413/06

(57) ABSTRACT

Provided herein is compounds of Formula I, methods of using the compounds as immunomodulators, and pharmaceutical compositions comprising such compounds. The compounds are useful in treating, preventing or ameliorating diseases or disorders such as cancer or infections.

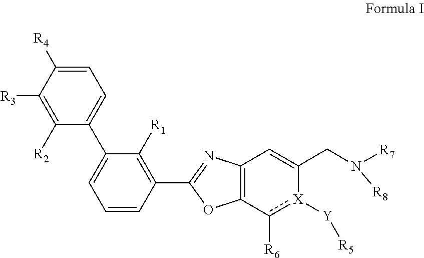

### FIELD OF THE INVENTION

The present application is concerned with pharmaceutically active compounds. The disclosure provides compounds as well as their compositions and methods of use. The compounds modulate PD-1/PD-L1 protein/protein interaction and are useful in the treatment of various diseases including infectious diseases and cancer.

### BACKGROUND OF THE INVENTION

The immune system plays an important role in controlling and eradicating diseases such as cancer. However, cancer cells often develop strategies to evade or to suppress the immune system in order to favor their growth. One such mechanism is altering the expression of co-stimulatory and co-inhibitory molecules expressed on immune cells (Postow et al, J. Clinical Oncology 2015, 1-9). Blocking the signaling of an inhibitory immune checkpoint, such as PD-1, has proven to be a promising and effective treatment modality.

The interaction between PD-1 and PD-L1 results in a decrease in tumor infiltrating lymphocytes, a decrease in T-cell receptor mediated proliferation, and immune evasion by the cancerous cells (Dong et al, J. Mol Med., 81:281-287 (2003); Blank et al, Cancer Immunol Immunother., 54:307-314 (2005); Konishi et al, Clin. Cancer Res. 10:5094-5100 (2004)). Immune suppression can be reversed by inhibiting the local interaction of PD-1 with PD-L1, and the effect is additive when the interaction of PD-1 with PD-L2 is blocked as well (Iwai et al., Proc. Natl. Acad. Sci. USA, 99: 12293-12297 (2002); Brown et al, J. Immunol, 170: 1257-1266 (2003)).

Programmed cell death-1 (PD-1), also known as CD279, is a cell surface receptor expressed on activated T cells, natural killer T cells, B cells, and macrophages (Greenwald et al, Annu. Rev. Immunol 2005, 23:515-548; Okazaki and Honjo, Trends Immunol 2006, (4): 195-201). It functions as an intrinsic negative feedback system to prevent the activation of T-cells, which in turn reduces autoimmunity and promotes self-tolerance. In addition, PD-1 is also known to play a critical role in the suppression of antigen-specific T cell response in diseases like cancer and viral infection (Sharpe et al, Nat Immunol 2007 8, 239-245; Postow et al, J. Clinical Oncol 2015, 1-9).

The structure of PD-1 consists of an extracellular immunoglobulin variable-like domain followed by a transmembrane region and an intracellular domain (Parry et al, Mol Cell Biol 2005, 9543-9553). The intracellular domain contains two phosphorylation sites located in an immunoreceptor tyrosine-based inhibitory motif and an immunoreceptor tyrosine-based switch motif, which suggests that PD-1 negatively regulates T cell receptor-mediated signals. PD-1 has two ligands, PD-L1 and PD-L2 (Parry et al, Mol Cell Biol 2005, 9543-9553; Latchman et al, Nat Immunol 2001, 2, 261-268), and they differ in their expression patterns. PD-L1 protein is upregulated on macrophages and dendritic cells in response to lipopolysaccharide and GM-CSF treatment, and on T cells and B cells upon T cell receptor and B cell receptor signaling. PD-L1 is also highly expressed on almost all tumor cells, and the expression is further increased after IFN-γ treatment (Iwai et al, PNAS2002, 99(19): 12293-7; Blank et al, Cancer Res 2004, 64(3): 1140-5). In fact, tumor PD-L1 expression status has been shown to be prognostic in multiple tumor types (Wang et al, Eur J Surg Oncol 2015; Huang et al, Oncol Rep 2015; Sabatier et al, Oncotarget 2015, 6(7): 5449-5464). PD-L2 expression, in contrast, is more restricted and is expressed mainly by dendritic cells (Nakae et al, J Immunol 2006, 177:566-73). Ligation of PD-1 with its ligands PD-L1 and PD-L2 on T cells delivers a signal that inhibits IL-2 and IFN-γ production, as well as cell proliferation induced upon T cell receptor activation (Carter et al, Eur J Immunol 2002, 32(3):634-43; Freeman et al, J Exp Med 2000, 192(7): 1027-34). The mechanism involves recruitment of SHP-2 or SHP-1 phosphatases to inhibit T cell receptor signaling such as Syk and Lck phosphorylation (Sharpe et al, Nat Immunol 2007, 8, 239-245). Activation of the PD-1 signaling axis also attenuates PKC-θ activation loop phosphorylation, which is necessary for the activation of NF-κB and API pathways, and for cytokine production such as IL-2, IFN-γ and TNF (Sharpe et al, Nat Immunol 2007, 8, 239-245; Carter et al, Eur J Immunol 2002, 32(3):634-43; Freeman et al, J Exp Med 2000, 192(7): 1027-34).

Several lines of evidence from preclinical animal studies indicate that PD-1 and its ligands negatively regulate immune responses. PD-1-deficient mice have been shown to develop lupus-like glomerulonephritis and dilated cardiomyopathy (Nishimura et al, Immunity 1999, 11:41-151; Nishimura et al, Science 2001, 291:319-322). Using an LCMV model of chronic infection, it has been shown that PD-1/PD-L1 interaction inhibits activation, expansion and acquisition of effector functions of virus-specific CD8 T cells (Barber et al, Nature 2006, 439, 682-7).

Together, these data support the development of a therapeutic approach to block the PD-1 mediated inhibitory signaling cascade in order to augment or “rescue” T cell response. Most of the currently approved medicines in immunotherapy are monoclonal antibodies. However, small molecule inhibitors that directly target PD-1 or PD-L1 are still not approved, there is only CA170 have been evaluated clinically.

Accordingly, there is still great demand for more potent, and more easily administered therapeutics against PD-1/PD-L1 protein/protein interactions. In this invention, applicant discovered potent small molecules that can have activity as inhibitors of the interaction of PD-L1 with PD-1, and thus may be useful for therapeutic administration to enhance immunity against cancer and/or infectious diseases. These small molecules are expected to be useful as pharmaceuticals with desirable stability, solubility, bioavailability, therapeutic index and toxicity values that are crucial to become efficient medicines to promote human health.

### SUMMARY OF INVENTION

The present invention relates to compounds that are used as inhibitors of the functional interaction between PD-L1 and PD-1. Inhibitors of the interaction between PD-L1 and PD-1 are useful in the treatment of cancers and infectious diseases.

The compounds of the invention have the general structures as Formula I. A compound of Formula I, or a stereoisomer, tautomer, pharmaceutically acceptable salt, prodrug, chelate, non-covalent complex, or solvate thereof,

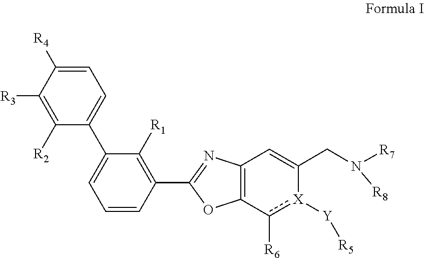

wherein,

X is selected from C, or N;

[symbol] is a single bond or a double bond.

R₁ is selected from H, halogen, CN, —C₁₋₈alkyl, —C₁₋₄haloalkyl, or —OC₁₋₈alkyl;

R₂, R₃ and R₄ are each independently selected from H, —OH, halogen, —CN, —C₁₋₈alkyl, —C₂₋₈alkenyl, —C₁₋₈alkoxyl, —O—C₁₋₄alkyl-C₅₋₁₀heterocycle, —C₃₋₁₀heteroaryl, —NHCO—C₈₋₁₀heteroaryl; —NHCO—C₁₋₄alkyl-C₅₋₁₀heterocycle; wherein —C₁₋₈alkyl, —C₁₋₈alkoxyl, —O—C₁₋₄alkyl-C₅₋₁₀heterocycle, —C₃₋₁₀heteroaryl, —NHCO—C₈₋₁₀heteroaryl, —NHCO—C₁₋₄alkyl-C₅₋₁₀heterocycle substituted with —C₁₋₈alkyl, —C₁₋₈alkoxyl, —C₃₋₁₀cycloalkyl, —C₃₋₁₀cycloalkyl-O—C₁₋₈alkyl; or

R₃ and R₄ together with the atoms to which they are attached form a 5- to 6-member heterocyclic ring; wherein the heterocyclic ring optionally comprising 1, 2 or 3 hetero atoms independently selected from N, S, or O;

Y is selected from absent, O, S, —NR₉—;

R₉ is selected from H, —C₁₋₈alkyl, or —C₁₋₈ haloalkyl;

R₅ is selected from H, halogen, CN, —C₁₋₈alkyl, —C₁₋₄haloalkyl, —C₂₋₈alkenyl, sulfonyl, sulfinyl, provided that if Y is O, then R₅ is not —C₁₋₈alkyl;

R₆ is H, or R₅ and R₆ together with the atoms to which they are attached form a 5- to 6-member heterocyclic ring; wherein the heterocyclic ring optionally comprising 1, 2 or 3 hetero atoms independently selected from N, S, or O; the heterocyclic ring optionally substituted with oxo, —C₁₋₈ alkyl, —C₀₋₄ alkyl-COOH, —C₀₋₄ alkyl-OH;

R₇ and R₈ are each independently selected from H, —C₁₋₈ alkyl, —C₁₋₆ alkyl-COOH, —C₅₋₆ aryl, wherein —C₁₋₆ alkyl-COOH and —C₅₋₆ aryl optionally substituted with —C₁₋₈ alkyl, —C₀₋₄ alkyl-COOH, —C₀₋₄ alkyl-OH; or

R₇ and R₈ together with the atoms to which they are attached form a 3- to 7-member heterocyclic ring; wherein the heterocyclic ring optionally comprising 1, 2 or 3 hetero atoms independently selected from N, S, or O; the heterocyclic ring optionally substituted with oxo, —C₁₋₈ alkyl, —C₀₋₄ alkyl-COOH, —C₀₋₄ alkyl-OH;

R₅ and R₈ together with the atoms to which they are attached form a 6- to 10-member heterocyclic ring; wherein the heterocyclic ring optionally comprising 1, 2 or 3 hetero atoms independently selected from N, S, or O; the heterocyclic ring optionally substituted with oxo, —C₁₋₈ alkyl, —C₀₋₄ alkyl-COOH, —C₀₋₄ alkyl-OH.

In some embodiments of Formula I, wherein Y is selected from absent, —O—, —S—, —NH—.

In some embodiments of Formula I, wherein R₁ is selected from CH₃ and CN.

In some embodiments of Formula I, wherein R₅ is selected from H, —CH₃, —CHF₂, —CF₃, —CH₂CF₃, F, Cl, CN,

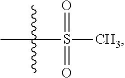 [SMILES: *S(C)(=O)=O]

and provided that if Y is O, then R₅ is not —CH₃.

The compound of Formula II, or a stereoisomer, tautomer, pharmaceutically acceptable salt, prodrug, chelate, non-covalent complex, or solvate thereof,

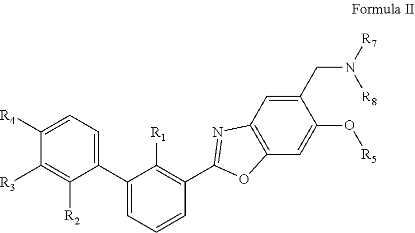

wherein,

R₁ is selected from H, halogen, CN, —C₁₋₈alkyl, —C₁₋₄haloalkyl, or —OC₁₋₈alkyl;

R₂, R₃ and R₄ are each independently selected from H, halogen, CN, —C₁₋₈alkyl, or —C₁₋₄haloalkyl;

R₅ is selected from H, —C₁₋₄haloalkyl, —SO₂—C₁₋₄ alkyl;

R₇ and R₈ are each independently selected from H, —C₁₋₈ alkyl, —C₁₋₆ alkyl-COOH, —C₅₋₆ aryl, wherein —C₁₋₆ alkyl-COOH and —C₅₋₆ aryl optionally substituted with —C₁₋₈ alkyl, —C₀₋₄ alkyl-COOH, —C₀₋₄ alkyl-OH; or

R₇ and R₈ together with the atoms to which they are attached form a 3- to 7-member heterocyclic ring; wherein the heterocyclic ring optionally comprising 1, 2 or 3 hetero atoms independently selected from N, S, or O; the heterocyclic ring optionally substituted with oxo, —C₁₋₈ alkyl, —C₀₋₄ alkyl-COOH, —C₀₋₄ alkyl-OH.

In some embodiments of Formula II, wherein R₁ is selected from CH₃ and CN.

In some embodiments of Formula II, wherein R₂, R₃ and R₄ are each independently selected from H, CN, or F.

In some embodiments of Formula II, wherein R₅ is —C₁₋₄haloalkyl.

In some embodiments of Formula II, wherein R₅ is —C₁₋₄alkyl substituted by 1-3 fluorine atoms.

In some embodiments of Formula II, wherein R₅ is methyl or ethyl substituted by 2-3 fluorine atoms.

In some embodiments of Formula II, wherein R₅ is —CF₃, —CHF₂, —CH₂CHF₂, or —CH₂CF₃.

In some embodiments of Formula II, wherein R₅ is —SO₂—C₁₋₄ alkyl.

In some embodiments of Formula II, wherein R₅ is —SO₂—CH₃.

In some embodiments of Formula II, wherein R₇ and R₈ are each independently selected from H, CH₃,

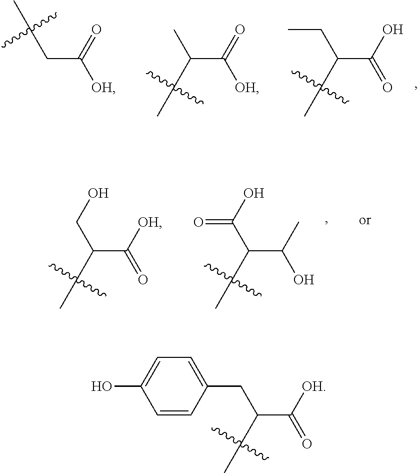

In some embodiments of Formula II, wherein R₇ and R₈ together with the atoms to which they are attached form a 4- to 6-member heterocyclic ring; wherein the heterocyclic ring substituted with —C₁₋₄ alkyl, —C₀₋₄ alkyl-COOH, or —C₀₋₄ alkyl-OH.

In some embodiments of Formula II, wherein heterocyclic ring is

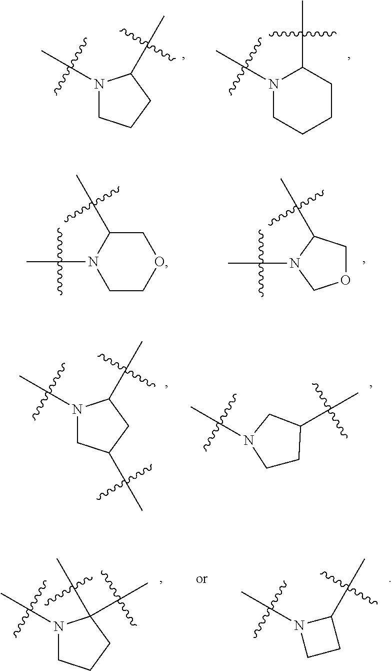

In some embodiments of Formula II, wherein the substituted heterocyclic ring is selected from

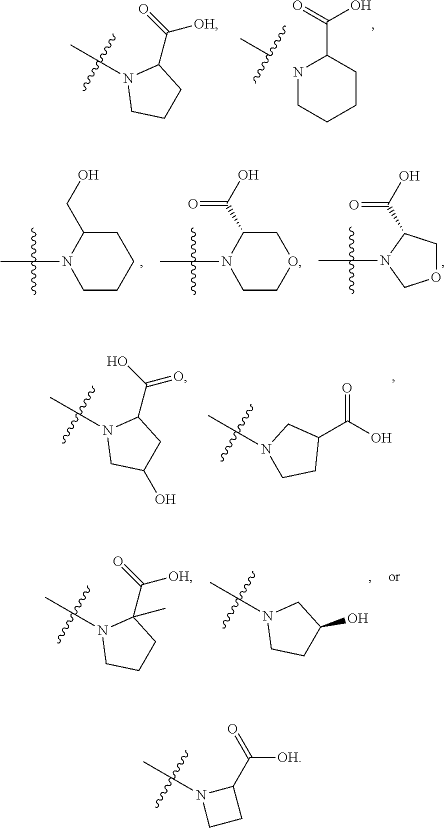

A compound of Formula III, or a stereoisomer, tautomer, pharmaceutically acceptable salt, prodrug, chelate, non-covalent complex, or solvate thereof,

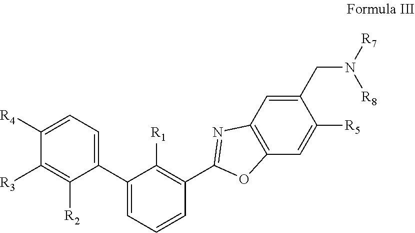

wherein,

R₁ is selected from H, halogen, CN, —C₁₋₈alkyl, —C₁₋₄haloalkyl, or —OC₁₋₈ alkyl;

R₂, R₃ and R₄ are each independently selected from H, alogen, CN, —C₁₋₈alkyl, or —C₁₋₄haloalkyl;

R₅ is selected from H, halogen, CN, —C₁₋₈alkyl, —C₁₋₄haloalkyl, —NH—C₁₋₄ alkyl, or —S—C₁₋₄ alkyl, sulfonyl, or sulfinyl;

R₇ and R₈ are each independently selected from H, —C₁₋₈ alkyl, —C₁₋₆ alkyl-COOH, —C₅₋₆ aryl, wherein —C₁₋₆ alkyl-COOH and —C₅₋₆ aryl optionally substituted with —C₁₋₈ alkyl, —C₀₋₄ alkyl-COOH, —C₀₋₄ alkyl-OH; or

R₇ and R₈ together with the atoms to which they are attached form a 3- to 7-member heterocyclic ring; wherein the heterocyclic ring optionally comprising 1, 2 or 3 hetero atoms independently selected from N, S, or O; the heterocyclic ring optionally substituted with oxo, —C₁₋₈ alkyl, —C₀₋₄ alkyl-COOH, —C₀₋₄ alkyl-OH.

In some embodiments of Formula III, R₁ is selected from CH₃, or CN.

In some embodiments of Formula III, wherein R₂, R₃ and R₄ are each independently selected from H, CN, or F.

In some embodiments of Formula III, wherein R₅ is selected from F, Cl, —CH₃, —CF₃, —S—CH₃, —SO—CH₃, —SO₂—CH₃, —CN, or —NHCH₃.

In some embodiments of Formula III, wherein R₇ and R₈ are each independently selected from

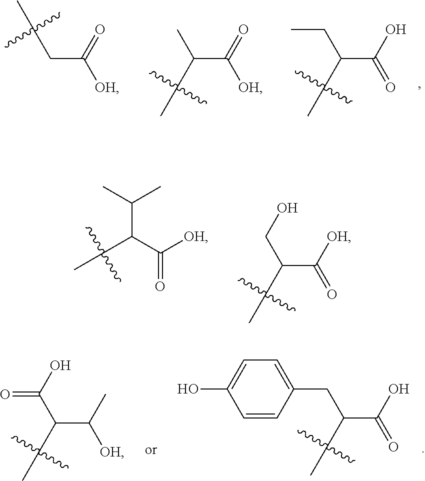

In some embodiments of Formula III, wherein R₇ and R₈ together with the atoms to which they are attached form a 5- to 6-member heterocyclic ring; wherein the heterocyclic ring substituted with —C₀₋₄ alkyl-COOH.

In some embodiments of Formula III, wherein the substituted heterocyclic ring is selected from

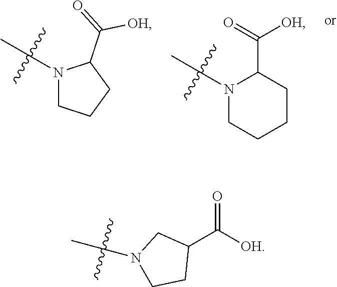 [SMILES: *N1CCCC1C(=O)O ; *N1CCCCC1C(=O)O ; *N1CCC(C(=O)O)C1]

The compound of Formula I, wherein the compound is
1) (S)-1-((2-(2-methyl-[1,1′-biphenyl]-3-yl)-6-(methylthio)benzo[d]oxazol-5-yl)methyl)piperidine-2-carboxylic acid;
2) ((2-(2-methyl-[1,1′-biphenyl]-3-yl)-6-(methylthio)benzo[d]oxazol-5-yl)methyl)-L-proline;
3) ((2-(2-methyl-[1,1′-biphenyl]-3-yl)-6-(methylthio)benzo[d]oxazol-5-yl)methyl)-D-allothreonine;
4) ((2-(2-methyl-[1,1′-biphenyl]-3-yl)-6-(methylthio)benzo[d]oxazol-5-yl)methyl)-L-allothreonine;
5) ((2-(2-methyl-[1,1′-biphenyl]-3-yl)-6-(methylthio)benzo[d]oxazol-5-yl)methyl)-L-alanine;
6) ((2-(2-methyl-[1,1′-biphenyl]-3-yl)-6-(methylthio)benzo[d]oxazol-5-yl)methyl)-D-alanine;
7) ((2-(2-methyl-[1,1′-biphenyl]-3-yl)-6-(methylthio)benzo[d]oxazol-5-yl)methyl)-D-proline;
8) (1-((2-(2-methyl-[1,1′-biphenyl]-3-yl)-6-(methylthio)benzo[d]oxazol-5-yl)methyl)piperidin-2-yl)methanol;
9) (S)-4-((2-(2-methyl-[1,1′-biphenyl]-3-yl)-6-(methylthio)benzo[d]oxazol-5-yl)methyl)morpholine-3-carboxylic acid;
10) (S)-2-(((2-(2-methyl-[1,1′-biphenyl]-3-yl)-6-(methylthio)benzo[d]oxazol-5-yl)methyl)amino)butanoic acid;
11) (R)-2-(((2-(2-methyl-[1,1′-biphenyl]-3-yl)-6-(methylthio)benzo[d]oxazol-5-yl)methyl)amino)butanoic acid;
12) ((2-(2-methyl-[1,1′-biphenyl]-3-yl)-6-(methylthio)benzo[d]oxazol-5-yl)methyl)-D-serine;
13) ((2-(2-methyl-[1,1′-biphenyl]-3-yl)-6-(methylthio)benzo[d]oxazol-5-yl)methyl)-L-serine;
14) ((2-(2-cyano-[1,1′-biphenyl]-3-yl)-6-(methylthio)benzo[d]oxazol-5-yl)methyl)-L-proline;
15) ((2-(2,2′-dicyano-[1,1′-biphenyl]-3-yl)-6-(methylthio)benzo[d]oxazol-5-yl)methyl)-L-proline;
16) 2-methyl-1-((2-(2-methyl-[1,1′-biphenyl]-3-yl)-6-(methylthio)benzo[d]oxazol-5-yl)methyl)pyrrolidine-2-carboxylic acid;
17) ((2-(2-cyano-[1,1′-biphenyl]-3-yl)-6-methylbenzo[d]oxazol-5-yl)methyl)-D-proline;
18) ((2-(2-cyano-[1,1′-biphenyl]-3-yl)-6-methylbenzo[d]oxazol-5-yl)methyl)-L-proline;
19) (S)-4-((2-(2-cyano-[1,1′-biphenyl]-3-yl)-6-methylbenzo[d]oxazol-5-yl)methyl)morpholine-3-carboxylic acid;
20) ((6-chloro-2-(2-methyl-[1,1′-biphenyl]-3-yl)benzo[d]oxazol-5-yl)methyl)-L-proline;
21) ((6-hydroxy-2-(2-methyl-[1,1′-biphenyl]-3-yl)benzo[d]oxazol-5-yl)methyl)-L-proline;
22) (S)-1-((6-methyl-2-(2-methyl-[1,1′-biphenyl]-3-yl)benzo[d]oxazol-5-yl)methyl)piperidine-2-carboxylic acid;
23) (S)-1-((6-fluoro-2-(2-methyl-[1,1′-biphenyl]-3-yl)benzo[d]oxazol-5-yl)methyl)pyrrolidin-3-ol;
24) ((6-fluoro-2-(2-methyl-[1,1′-biphenyl]-3-yl)benzo[d]oxazol-5-yl)methyl)-L-proline;
25) (S)-1-((6-fluoro-2-(2-methyl-[1,1′-biphenyl]-3-yl)benzo[d]oxazol-5-yl)methyl)piperidine-2-carboxylic acid;
26) 1-((6-cyano-2-(2-methyl-[1,1′-biphenyl]-3-yl)benzo[d]oxazol-5-yl)methyl)piperidine-2-carboxylic acid;
27) 1-((2-(2-methyl-[1,1′-biphenyl]-3-yl)-6-(methylamino)benzo[d]oxazol-5-yl)methyl)piperidine-2-carboxylic acid;
28) ((2-(2-methyl-[1,1′-biphenyl]-3-yl)-6-(trifluoromethyl)benzo[d]oxazol-5-yl)methyl)-L-proline;
29) (S)-1-((6-(difluoromethoxy)-2-(2-methyl-[1,1′-biphenyl]-3-yl)benzo[d]oxazol-5-yl)methyl)piperidine-2-carboxylic acid;
30) ((6-(difluoromethoxy)-2-(2-methyl-[1,1′-biphenyl]-3-yl)benzo[d]oxazol-5-yl)methyl)-D-allothreonine;
31) ((6-(difluoromethoxy)-2-(2-methyl-[1,1′-biphenyl]-3-yl)benzo[d]oxazol-5-yl)methyl)-L-allothreonine;
32) ((6-(difluoromethoxy)-2-(2-methyl-[1,1′-biphenyl]-3-yl)benzo[d]oxazol-5-yl)methyl)-L-tyrosine hydrochloride;
33) ((6-(difluoromethoxy)-2-(2-methyl-[1,1′-biphenyl]-3-yl)benzo[d]oxazol-5-yl)methyl)-L-alanine;
34) ((6-(difluoromethoxy)-2-(2-methyl-[1,1′-biphenyl]-3-yl)benzo[d]oxazol-5-yl)methyl)-D-alanine;
35) ((6-(difluoromethoxy)-2-(2-methyl-[1,1′-biphenyl]-3-yl)benzo[d]oxazol-5-yl)methyl)glycine;
36) ((6-(difluoromethoxy)-2-(2-methyl-[1,1′-biphenyl]-3-yl)benzo[d]oxazol-5-yl)methyl)-L-proline;
37) ((6-(difluoromethoxy)-2-(2-methyl-[1,1′-biphenyl]-3-yl)benzo[d]oxazol-5-yl)methyl)-D-proline;
38) (1-((6-(difluoromethoxy)-2-(2-methyl-[1,1′-biphenyl]-3-yl)benzo[d]oxazol-5-yl)methyl)piperidin-2-yl)methanol;
39) (S)-4-((6-(difluoromethoxy)-2-(2-methyl-[1,1′-biphenyl]-3-yl)benzo[d]oxazol-5-yl)methyl)morpholine-3-carboxylic acid;
40) (S)-2-(((6-(difluoromethoxy)-2-(2-methyl-[1,1′-biphenyl]-3-yl)benzo[d]oxazol-5-yl)methyl)amino)butanoic acid;
41) (R)-2-(((6-(difluoromethoxy)-2-(2-methyl-[1,1′-biphenyl]-3-yl)benzo[d]oxazol-5-yl)methyl)amino)butanoic acid;
42) ((6-(difluoromethoxy)-2-(2-methyl-[1,1′-biphenyl]-3-yl)benzo[d]oxazol-5-yl)methyl)-D-serine;
43) ((6-(difluoromethoxy)-2-(2-methyl-[1,1′-biphenyl]-3-yl)benzo[d]oxazol-5-yl)methyl)-L-serine;
44) ((2-(2-cyano-[1,1′-biphenyl]-3-yl)-6-(difluoromethoxy)benzo[d]oxazol-5-yl)methyl)-L-proline;
45) ((2-(2,2′-dicyano-[1,1′-biphenyl]-3-yl)-6-(difluoromethoxy)benzol[d]oxazol-5-yl)methyl)-L-proline;
46) 1-((6-(difluoromethoxy)-2-(2-methyl-[1,1′-biphenyl]-3-yl)benzo[d]oxazol-5-yl)methyl)-2-methylpyrrolidine-2-carboxylic acid;
47) ((6-(difluoromethoxy)-2-(3-(2,3-dihydrobenzo[b][1,4]dioxin-6-yl)-2-methylphenyl)benzo[d]oxazol-5-yl)methyl)-L-proline;
48) 1-((6-(difluoromethoxy)-2-(2-methyl-[1,1′-biphenyl]-3-yl)benzo[d]oxazol-5-yl)methyl)azetidine-2-carboxylic acid;
49) ((6-(difluoromethoxy)-2-(3-(2,3-dihydrobenzo[b][1,4]dioxin-6-yl)-2-methylphenyl)benzo[d]oxazol-5-yl)methyl)-L-proline;
50) 1-((6-(difluoromethoxy)-2-(2-methyl-[1,1′-biphenyl]-3-yl)benzo[d]oxazol-5-yl)methyl)-2-methylpyrrolidine-2-carboxylic acid;
51) ((2-(2′-cyano-2-methyl-[1,1′-biphenyl]-3-yl)-6-(difluoromethoxy)benzo[d]oxazol-5-yl)methyl)proline;
52) ((6-(difluoromethoxy)-2-(2′-fluoro-2-methyl-[1,1′-biphenyl]-3-yl)benzo[d]oxazol-5-yl)methyl)-L-proline;
53) (S)-3-((6-(difluoromethoxy)-2-(2-methyl-[1,1′-biphenyl]-3-yl)benzo[d]oxazol-5-yl)methyl)oxazolidine-4-carboxylic acid;
54) (S)-1-((2-(2-methyl-[1,1′-biphenyl]-3-yl)-6-(2,2,2-trifluoroethoxy)benzo[d]oxazol-5-yl)methyl)piperidine-2-carboxylic acid;
55) 1-((6-(2,2-difluoroethoxy)-2-(2-methyl-[1,1′-biphenyl]-3-yl)benzo[d]oxazol-5-yl)methyl)piperidine-2-carboxylic acid;
56) ((2-(2-methyl-[1,1′-biphenyl]-3-yl)-6-(2,2,2-trifluoroethoxy)benzo[d]oxazol-5-yl)methyl)-D-alanine;
57) (S)-4-((2-(2-cyano-[1,1′-biphenyl]-3-yl)-6-(2,2,2-trifluoroethoxy)benzo[d]oxazol-5-yl)methyl)morpholine-3-carboxylic acid;
58) (S)-4-((2-(2-methyl-[1,1′-biphenyl]-3-yl)-6-(2,2,2-trifluoroethoxy)benzo[d]oxazol-5-yl)methyl)morpholine-3-carboxylic acid;
59) ((2-(2-methyl-[1,1′-biphenyl]-3-yl)-6-(2,2,2-trifluoroethoxy)benzo[d]oxazol-5-yl)methyl)-L-alanine;
60) ((2-(2-methyl-[1,1′-biphenyl]-3-yl)-6-(2,2,2-trifluoroethoxy)benzo[d]oxazol-5-yl)methyl)-L-proline;
61) 2-methyl-1-((2-(2-methyl-[1,1′-biphenyl]-3-yl)-6-(2,2,2-trifluoroethoxy)benzo[d]oxazol-5-yl)methyl)pyrrolidine-2-carboxylic acid;
62) ((2-(2-methyl-[1,1′-biphenyl]-3-yl)-6-(2,2,2-trifluoroethoxy)benzo[d]oxazol-5-yl)methyl)-D-alanine;
63) ((2-(2-methyl-[1,1′-biphenyl]-3-yl)-6-(trifluoromethoxy)benzo[d]oxazol-5-yl)methyl)-L-proline;
64) ((2-(2-methyl-[1,1′-biphenyl]-3-yl)-6-((methylsulfonyl)oxy)benzo[d]oxazol-5-yl)methyl)-L-proline;
65) ((2-(2-methyl-[1,1′-biphenyl]-3-yl)-6-(methylsulfinyl)benzo[d]oxazol-5-yl)methyl)-L-proline;
66) ((2-(2-methyl-[1,1′-biphenyl]-3-yl)-6-(methylsulfonyl)benzo[d]oxazol-5-yl)methyl)-L-proline;
67) 8-methyl-2-(2-methyl-[1,1′-biphenyl]-3-yl)-6,7,8,9-tetrahydrooxazolo[5′,4′:4,5]benzo[1,2-f][1,4]oxazepine;
68) 1-((2-(2-methyl-[1,1′-biphenyl]-3-yl)-8-oxo-7,8-dihydrobenzofuro[5,4-d]oxazol-5-yl)methyl)piperidine-2-carboxylic acid;
69) 1-((8-(2-methyl-[1,1′-biphenyl]-3-yl)oxazolo[5,4-c][1,2,4]triazolo[1,5-a]pyridin-5-yl)methyl)piperidine-2-carboxylic acid;
70) (S)-2-(2-methyl-[1,1′-biphenyl]-3-yl)-6,7,8,9-tetrahydrooxazolo[5′,4′:4,5]benzo[1,2-f][1,4]oxazepine-7-carboxylic acid;
71) 2-(2-(2-methyl-[1,1′-biphenyl]-3-yl)-6,7-dihydrooxazolo[5′,4′:4,5]benzo[1,2-f][1,4]oxazepin-8(9H)-yl)acetic acid;
72) (R)-2-(2-methyl-[1,1′-biphenyl]-3-yl)-6,7,8,9-tetrahydrooxazolo[5′,4′:4,5]benzo[1,2-f][1,4]oxazepine-7-carboxylic acid;
73) 1-((2-(2-methyl-[1,1′-biphenyl]-3-yl)-8-oxo-7,8-dihydrobenzofuro[5,4-d]oxazol-5-yl)methyl)piperidine-2-carboxylic acid.

The present invention also provides a pharmaceutical composition comprising a compound of any of the present invention and a pharmaceutically acceptable excipient. Such as hydroxypropyl methyl cellulose. In the composition, the said compound in a weight ratio to the said excipient within the range from about 0.0001 to about 10.

The present invention additionally provided a use of a pharmaceutical composition of Formula I for the preparation of a medicament for treating a disease in a subject.

The present invention further provides some preferred technical solutions with regard to above-mentioned uses.

In some embodiments, a medicament thus prepared can be used for the treatment or prevention of, or for delaying or preventing onset or progression in, cancer, cancer metastasis, an immunological disorder. The cancer is colon cancer, gastric cancer, thyroid cancer, lung cancer, leukemia, pancreatic cancer, melanoma, multiple melanoma, brain cancer, renal cancer, prostate cancer, ovarian cancer or breast cancer.

The present invention provided a method of inhibiting PD-1/PD-L1 interaction, said method comprising administering to a patient a compound of any one of claims 1-12, or a pharmaceutically acceptable salt or a stereoisomer thereof.

The present invention provided a method of treating a disease associated with inhibition of PD-1/PD-L1 interaction, said method comprising administering to a patient in need thereof a therapeutically effective amount of a compound of the present invention, or a pharmaceutically acceptable salt or a stereoisomer thereof. Wherein the disease is colon cancer, gastric cancer, thyroid cancer, lung cancer, leukemia, pancreatic cancer, melanoma, multiple melanoma, brain cancer, renal cancer, prostate cancer, ovarian cancer or breast cancer.

The present invention provided a method of enhancing, stimulating and/or increasing the immune response in a patient, said method comprising administering to the patient in need thereof a therapeutically effective amount of a compound of the present invention, or a pharmaceutically acceptable salt or a stereoisomer thereof.

The present invention also provides a use of the present compound or its pharmaceutical composition for the preparation of a medicament.

In some embodiments, the medicament is used for the treatment or prevention of cancer.

In some embodiments, the cancer is colon cancer, gastric cancer, thyroid cancer, lung cancer, leukemia, pancreatic cancer, melanoma, multiple melanoma, brain cancer, renal cancer, prostate cancer, ovarian cancer or breast cancer.

In some embodiments, the medicament is used as an inhibitor of PD-1/PD-L1 interaction.

The general chemical terms used in the formula above have their usual meanings. For example, the term “halogen”, as used herein, unless otherwise indicated, means fluoro, chloro, bromo or iodo. The preferred halogen groups include F, Cl and Br.

As used herein, unless otherwise indicated, alkyl includes saturated monovalent hydrocarbon radicals having straight, branched or cyclic moieties. For example, alkyl radicals include methyl, ethyl, propyl, isopropyl, cyclcopropyl, n-butyl, isobutyl, sec-butyl, t-butyl, cyclcobutyl, n-pentyl, 3- (2-methyl) butyl, 2-pentyl, 2-methylbutyl, neopentyl, cyclcopentyl, n-hexyl, 2-hexyl, 2-methylpentyl and cyclohexyl. Similarly, C₁₋₈, as in C₁₋₈alkyl is defined to identify the group as having 1, 2, 3, 4, 5, 6, 7 or 8 carbon atoms in a linear or branched arrangement.

Alkenyl and alkynyl groups include straight, branched chain or cyclic alkenes and alkynes. Likewise, “C₂₋₈ alkenyl” and “C₂₋₈ alkynyl” means an alkenyl or alkynyl radicals having 2, 3, 4, 5, 6, 7 or 8 carbon atoms in a linear or branched arrangement.

Alkoxy radicals are oxygen ethers formed from the previously described straight, branched chain or cyclic alkyl groups.

The term “aryl”, as used herein, unless otherwise indicated, refers to an unsubstituted or substituted mono- or polycyclic ring system containing carbon ring atoms. The preferred aryls are mono cyclic or bicyclic 6-10 membered aromatic ring systems. Phenyl and naphthyl are preferred aryls. The most preferred aryl is phenyl.

The term “heterocyclyl”, as used herein, unless otherwise indicated, represents an unsubstituted or substituted stable three to eight membered monocyclic saturated ring system which consists of carbon atoms and from one to three heteroatoms selected from N, O or S, and wherein the nitrogen or sulfur heteroatoms may optionally be oxidized, and the nitrogen heteroatom may optionally be quaternized. The heterocyclyl group may be attached at any heteroatom or carbon atom which results in the creation of a stable structure. Examples of such heterocyclyl groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinyl sulfoxide, thiamorpholinyl sulfone and oxadiazolyl.

The term “heteroaryl”, as used herein, unless otherwise indicated, represents an unsubstituted or substituted stable five or six membered monocyclic aromatic ring system or an unsubstituted or substituted nine or ten membered benzo-fused heteroaromatic ring system or bicyclic heteroaromatic ring system which consists of carbon atoms and from one to four heteroatoms selected from N, O or S, and wherein the nitrogen or sulfur heteroatoms may optionally be oxidized, and the nitrogen heteroatom may optionally be quaternized. The heteroaryl group may be attached at any heteroatom or carbon atom which results in the creation of a stable structure. Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adenylyl, quinolinyl or isoquinolinyl.

The term “alkenyloxy” refers to the group —O-alkenyl, where alkenyl is defined as above.

The term “alknyloxy” refers to the group —O-alknyl, where alknyl is defined as above.

The term “cycloalkyl” to a cyclic saturated alkyl chain having from 3 to 12 carbon atoms, for example, cyclopropyl, cyclobutyl, cyclobutyl, cyclobutyl.

The term “substituted” refers to a group in which one or more hydrogen atoms are each independently replaced with the same or different substituent(s). Typical substituents include, but are not limited to, halogen (F, Cl, Br or I), C₁₋₈ alkyl, C₃₋₁₂ cycloalkyl, —OR¹, SR¹, ═O, ═S, —C(O)R¹, —C(S)R¹, ═NR¹, —C(O)OR¹, —C(S)OR¹, —NR¹R², —C(O)NR¹R², cyano, nitro, —S(O)₂R¹, —OS(O₂)OR¹, —OS(O)₂R¹, —OP(O)(OR¹)(OR²); wherein R¹ and R² is independently selected from —H, lower alkyl, lower haloalkyl. In some embodiments, the substituent(s) is independently selected from the group consisting of —F, —Cl, —Br, —I, —OH, trifluoromethoxy, ethoxy, propyloxy, iso-propyloxy, n-butyloxy, isobutyloxy, t-butyloxy, —SCH₃, —SC₂H₅, formaldehyde group, —C(OCH₃), cyano, nitro, CF₃, —OCF₃, amino, dimethylamino, methyl thio, sulfonyl and acetyl.

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

Examples of substituted alkyl group include, but not limited to, 2-aminoethyl, 2-hydroxyethyl, pentachloroethyl, trifluoromethyl, methoxymethyl, pentafluoroethyl and piperazinylmethyl.

Examples of substituted alkoxy groups include, but not limited to, aminomethoxy, trifluoromethoxy, 2-diethylaminoethoxy, 2-ethoxycarbonylmethoxy, 3-hydroxypropoxy.

The compounds of the present invention may also be present in the form of pharmaceutically acceptable salts. For use in medicine, the salts of the compounds of this invention refer to non-toxic “pharmaceutically acceptable salts”. The pharmaceutically acceptable salt forms include pharmaceutically acceptable acidic/anionic or basic/cationic salts. The pharmaceutically acceptable acidic/anionic salt generally takes a form in which the basic nitrogen is protonated with an inorganic or organic acid. Representative organic or inorganic acids include hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic. Pharmaceutically acceptable basic/cationic salts include, and are not limited to aluminum, calcium, chloroprocaine, choline, diethanolamine, ethylenediamine, lithium, magnesium, potassium, sodium and zinc.

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The above Formula I are shown without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of Formula I exists, the present invention includes any possible tautomers and pharmaceutically acceptable salts thereof, and mixtures thereof, except where specifically stated otherwise.

When the compound of Formula I and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids, particularly preferred are formic and hydrochloric acid. Since the compounds of Formula I are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or a prodrug, or a metabolite, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound, or a pharmaceutically acceptable salt, of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include such as lactose, terra alba, sucrose, talc, gelatin, agar, pectin, acacia, magnesium stearate, and stearic acid. Examples of liquid carriers include such as sugar syrup, peanut oil, olive oil, and water. Examples of gaseous carriers include such as carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, colon cancer, rectal cancer, mantle cell lymphoma, multiple myeloma, breast cancer, prostate cancer, glioblastoma, squamous cell esophageal cancer, liposarcoma, T-cell lymphoma melanoma, pancreatic cancer, glioblastoma or lung cancer, may be effectively treated by the administration of from about 0.01 to 50 mg of the compound per kilogram of body weight per day, or alternatively about 0.5 mg to about 3.5 g per patient per day.

It is understood, however, that lower or higher doses than those recited above may be required. Specific dose level and treatment regimens for any particular subject will depend upon a variety of factors, including the activity of the specific compound employed, the age, body weight, general health, sex, diet, time of administration, route of administration, rate of excretion, drug combination, the severity and course of the particular disease undergoing therapy, the subject disposition to the disease, and the judgment of the treating physician.

These and other aspects will become apparent from the following written description of the invention.

The following Examples are provided to better illustrate the present invention. All parts and percentages are by weight and all temperatures are degrees Celsius, unless explicitly stated otherwise.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to inhibit the activity of PD-1/PD-L1 protein/protein interaction according to at least one assay described herein.

### EXAMPLES

Experimental procedures for compounds of the invention are provided below. Open Access Preparative LCMS Purification of some of the compounds prepared was performed on Waters mass directed fractionation systems. The basic equipment setup, protocols and control software for the operation of these systems have been described in detail in literature. See, e.g., Blom, “Two-Pump At Column Dilution Configuration for Preparative LC-MS”, K. Blom, J. Combi. Chem., 2002, 4, 295-301; Blom et al, “Optimizing Preparative LC-MS Configurations and Methods for Parallel Synthesis Purification”, J. Combi. Chem., 2003, 5, 670-83; and Blom et al., “Preparative LC-MS Purification: Improved Compound Specific Method Optimization”, J. Combi. Chem., 2004, 6, 874-883.

The compounds described herein can be obtained from commercial sources or synthesized by conventional methods as shown below using commercially available starting materials and reagents. The following abbreviations have been used in the examples:

AcOH or HOAC: Ethanoic acid;

BSA: Bovine serum album;

DCM: Dichloromethane;

DDQ: 2,3-Dichloro-5,6-dicyano-p-benzoquinone;

DMSO: Dimethyl sulfoxide;

EtOAc: Ethyl acetate;

h or hrs: hour or hours;

HTRF: Homogeneous Time Resolved Fluorescence;

MeOH: Methanol

min: minute;

PE: petroleum ether;

Pd(dppf)Cl₂: [1,1′-Bis(diphenylphosphino)ferrocene]dichloropalladium;

rt or r.t.: room temperature;

TBAI: Tetrabutylammonium Iodide

THF: Tetrahydrofuran.

### Example 1 Synthesis of Compound 1

### (S)-1-((2-(2-methyl-[1,1′-biphenyl]-3-yl)-6-(methylthio)benzo[d]oxazol-5-yl)methyl)piperidine-2-carboxylic acid

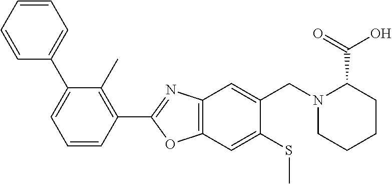 [SMILES: CSc1cc2oc(-c3cccc(-c4ccccc4)c3C)nc2cc1CN1CCCC[C@H]1C(=O)O]

### Step 1: Preparation of methyl 4-hydroxy-2-(methylthio)-5-nitrobenzoate

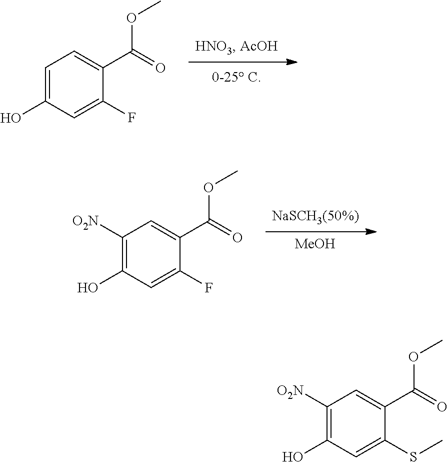 [SMILES: "MeOH" ; COC(=O)c1ccc(O)cc1F ; COC(=O)c1cc([N+](=O)[O-])c(O)cc1F ; COC(=O)c1cc([N+](=O)[O-])c(O)cc1SC]

Methyl 2-fluoro-4-hydroxybenzoate (20.00 g) was dissolved in 200 mL acetic acid, cooled to 0-10° C. in an ice bath, and then concentrated nitric acid (10.1 ml, 0.236 mol) was dissolved in 40 mL acetic acid and slowly added dropwise to the above reaction solution; after the addition, removed the ice bath and naturally raised to room temperature and stirred for 4-6 h. The reaction solution was poured into ice water and quenched. After stirring for 0.5 h, the solid was completely precipitated, filtered, and the filter cake was washed with water 2-3 times, dried, and the crude product was purified by flash chromatography (A.hexane; B.EA; B % from 0-30%, 20 min) gave the product 13.00 g methyl 2-fluoro-4-hydroxy-5-nitrobenzoate as a light yellow solid.

A mixture of methyl 2-fluoro-4-hydroxy-5-nitrobenzoate (8.00 g) and Sodium thiomethoxide solid (50%)(15.56 g) in methanol (80 ml) was stirred at room temperature, then stirred at 80° C. for 20 h. The solid residue was obtained by filtration, and then dried under vacuum to obtain 7.60 g crude product methyl 4-hydroxy-2-(methylthio)-5-nitrobenzoate.

### Step 2: Preparation of methyl 5-amino-2,4-dihydroxybenzoate

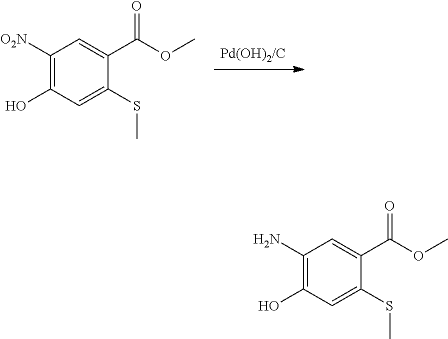 [SMILES: COC(=O)c1cc([N+](=O)[O-])c(O)cc1SC ; COC(=O)c1cc(N)c(O)cc1SC]

A mixture of methyl 4-hydroxy-2-(methylthio)-5-nitrobenzoate (77.1 g) and 10% Pd/C (11.5 g) in methanol (2 L) was stirred under 1.1 atm of hydrogen pressure at room temperature for 3 h. The catalyst was removed by filtration, the solid residue was washed with methanol (300 mL) and then dried under vacuum to obtain 72 g crude product methyl 5-amino-4-hydroxy-2-(methylthio)benzoate.

### Step 3: Preparation of methyl 2-(3-bromo-2-methylphenyl)-6-(methylthio)benzo[d]oxazole-5-carboxylate

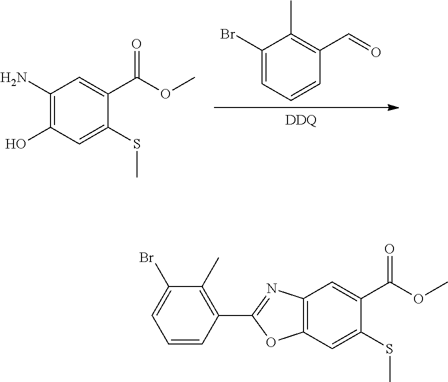

A mixture of methyl 5-amino-4-hydroxy-2-(methylthio)benzoate (32.9 g) and 3-bromo-2-methylbenzaldehyde (32.5 g) in MeOH (1 L) was stirred for 2.5 h at 80° C., then the mixture was concentered under reduced pressure. The mixture was added DCM (500 ml) and DDQ (55.6 g). The mixture was stirred at room temperature for 1 h. The reaction was diluted with DCM and washed with an aqueous Na₂S₂O₃ solution and NaHCO₃ solution. The organic phase was dried over MgSO₄, filtered and the filtrate was concentrated. The crude product was purified by column chromatography (PE:DCM=1:1) to afford 45 g methyl 2-(3-bromo-2-methylphenyl)-6-(methylthio)benzo[d]oxazole-5-carboxylate as a brown solid.

### Step 4: Preparation of methyl 2-(2-methyl-[1,1′-biphenyl]-3-yl)-6-(methylthio)benzo[d]oxazole-5-carboxylate

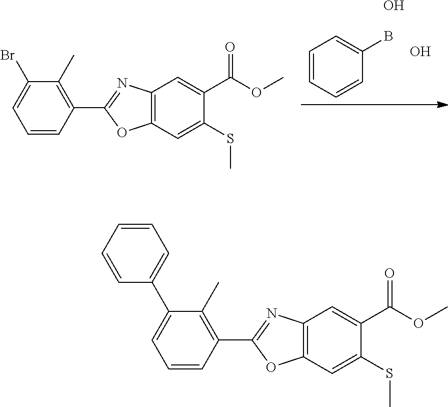

Methyl 2-(3-bromo-2-methylphenyl)-6-(methylthio)benzo[d]oxazole-5-carboxylate (6.00 g), phenylboronic acid (2.70 g), K₂CO₃(6.10 g), Pd(dppf)Cl₂(1.10 g) was added to dioxiane (50 mL) under nitrogen at 80° C. for 120 min. The mixture was cooled and diluted with DCM, then washed with H₂O and NaCl solution. The organic phase was dried over MgSO₄, filtered and the filtrate was concentrated. The crude product was purified by column chromatography (PE:DCM=1:5) to afford 5 g methyl 2-(2-methyl-[1,1′-biphenyl]-3-yl)-6-(methylthio)benzo[d]oxazole-5-carboxylate.

### Step 5: Preparation of (2-(2-methyl-[1,1′-biphenyl]-3-yl)-6-(methylthio)benzo[d]oxazol-5-yl)methanol

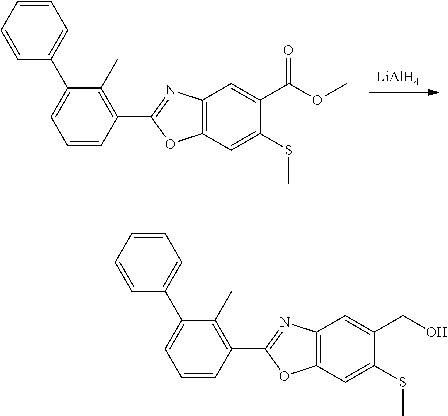 [SMILES: COC(=O)c1cc2nc(-c3cccc(-c4ccccc4)c3C)oc2cc1SC ; "LiAlH4" ; CSc1cc2oc(-c3cccc(-c4ccccc4)c3C)nc2cc1CO]

To a solution of methyl 2-(2-methyl-[1,1′-biphenyl]-3-yl)-6-(methylthio)benzo[d]oxazole-5-carboxylate (1.30 g) and THF (50 mL) was added LiAlH4 in THF (2.5 M, 5 mL) dropwise at 0° C., The mixture was warmed up to room temperature. After 1 h, the mixture was quenched with 1 mL H₂O and 1 mL 10% NaOH solution, then washed with 1 M HCl, water and brine separately. The organic phase was dried over Na₂SO₄, filtered and concentrated the filtrate to obtain (2-(2-methyl-[1,1′-biphenyl]-3-yl)-6-(methylthio)benzo[d]oxazol-5-yl)methanol as a yellow solid (1.2 g).

### Step 6: Preparation of 2-(2-methyl-[1,1′-biphenyl]-3-yl)-6-(methylthio)benzo[d]oxazole-5-carbaldehyde

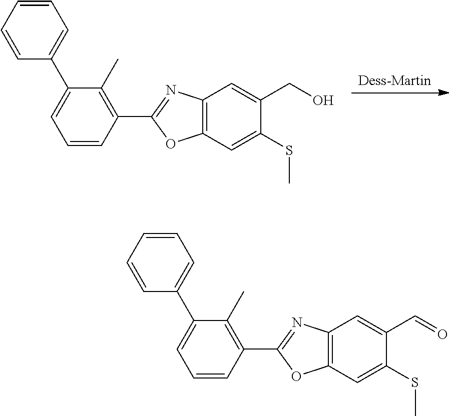 [SMILES: CSc1cc2oc(-c3cccc(-c4ccccc4)c3C)nc2cc1CO ; "Dess-Martin" ; CSc1cc2oc(-c3cccc(-c4ccccc4)c3C)nc2cc1C=O]

To a solution of (2-(2-methyl-[1,1′-biphenyl]-3-yl)-6-(methylthio)benzo[d]oxazol-5-yl)methanol (1.40 g) in dry THF (15 mL) was added Dess-Martin (2.39 g) at 10° C. Then the mixture solution was stirred for 1 h at room temperature. The mixture was filtered through Celite. The solid was washed with DCM, and the combined filtrate was washed with NaHCO₃ aqueous solution, water and brine separately, dried and concentrated. The residue was purified by column chromatography (eluting with hexane-EtOAc using a gradient from 20:1 to 5:1) to afford 2-(2-methyl-[1,1′-biphenyl]-3-yl)-6-(methylthio)benzo[d]oxazole-5-carbaldehyde as solid (1.27 g).

### Step 7: Preparation of (S)-1-((2-(2-methyl-[1,1′-biphenyl]-3-yl)-6-(methylthio)benzo[d]oxazol-5-yl)methyl)piperidine-2-carboxylic acid

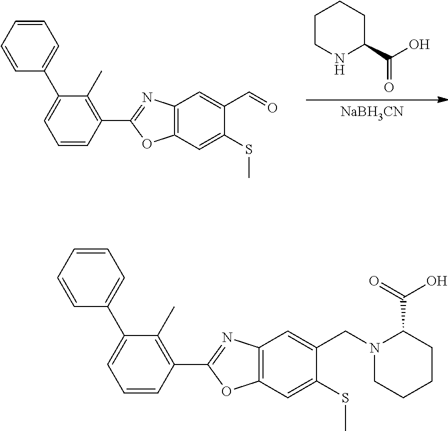

A mixture of 2-(2-methyl-[1,1′-biphenyl]-3-yl)-6-(methylthio)benzo[d]oxazole-5-carbaldehyde (1.00 g), (S)-piperidine-2-carboxylic acid (1.70 g), HOAC (316 mg) in MeOH was stirred at room temperature for 0.5 hrs. Then NaBH₃CN (498 mg) was added, heated at 60° C. for 2 h. The mixture was cooled and diluted with DCM and then washed with H₂O and NaCl solution separately. The organic phase was dried over MgSO₄, filtered and concentrated the filtrate. The residue was purified by column chromatography (DCM:MeOH=8%), to afford (S)-1-((2-(2-methyl-[1,1′-biphenyl]-3-yl)-6-(methylthio)benzo[d]oxazol-5-yl)methyl)piperidine-2-carboxylic acid as a white solid. (671 mg).

### Example 2 Synthesis of Compound 2

### ((2-(2-methyl-[1,1′-biphenyl]-3-yl)-6-(methylthio)benzo[d]oxazol-5-yl)methyl)-L-proline

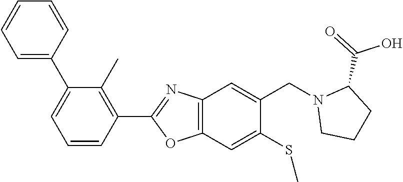 [SMILES: CSc1cc2oc(-c3cccc(-c4ccccc4)c3C)nc2cc1CN1CCC[C@H]1C(=O)O]

Prepare the Compound 2 essentially as described for Example 1 using the corresponding intermediate. For example, using “L-proline” instead of “(S)-piperidine-2-carboxylic acid” in the last step (step 7) described above.

Prepare the following examples (shown in Table 1) essentially as described for Example 1 using

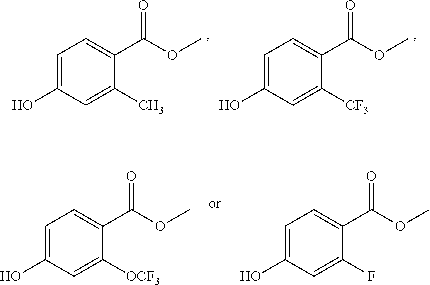 [SMILES: COC(=O)c1ccc(O)cc1C ; COC(=O)c1ccc(O)cc1C(F)(F)F ; COC(=O)c1ccc(O)cc1OC(F)(F)F ; COC(=O)c1ccc(O)cc1F]

instead of

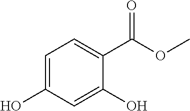

in the step 1 of Example 29, in some examples, using other amide acid, for example L-proline, instead of (S)-piperidine-2-carboxylic acid in the step 7 described above.

TABLE 1

| | | | Physical |
|---|---|---|---|
| EX | | | Data (MS) |
| No. | Chemical Name | Structure | (M + H)⁺ |
| 1 | (S)-1-((2-(2-methyl-[1,1′-biphenyl]- 3-yl)-6- (methylthio)benzo[d]oxazol-5- yl)methyl)piperidine-2-carboxylic acid | 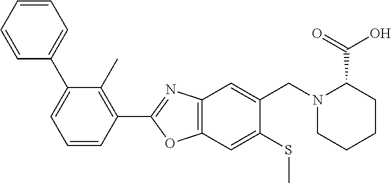 [SMILES: CSc1cc2oc(-c3cccc(-c4ccccc4)c3C)nc2cc1CN1CCCC[C@H]1C(=O)O] | 473.2 |
| 2 | ((2-(2-methyl-[1,1′-biphenyl]-3-yl)- 6-(methylthio)benzo[d]oxazol-5- yl)methyl)-L-proline | 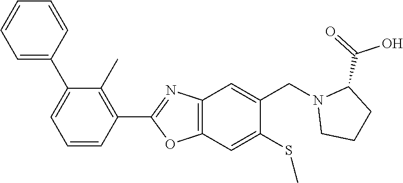 [SMILES: CSc1cc2oc(-c3cccc(-c4ccccc4)c3C)nc2cc1CN1CCC[C@H]1C(=O)O] | 459.2 |
| 3 | ((2-(2-methyl-[1,1′-biphenyl]-3-yl)- 6-(methylthio)benzo[d]oxazol-5- yl)methyl)-D-allothreonine | 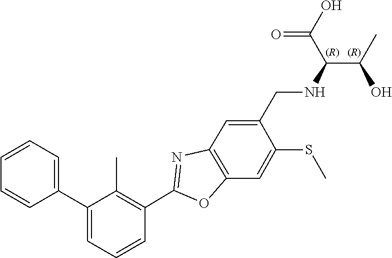 | 463.2 |
| 4 | ((2-(2-methyl-[1,1′-biphenyl]-3-yl)- 6-(methylthio)benzo[d]oxazol-5- yl)methyl)-L-allothreonine | 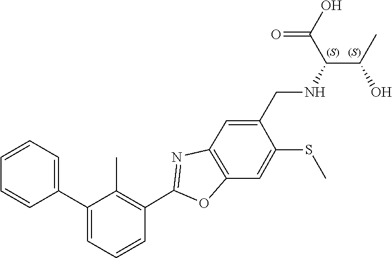 | 463.2 |
| 5 | ((2-(2-methyl-[1,1′-biphenyl]-3-yl)- 6-(methylthio)benzo[d]oxazol-5- yl)methyl)-L-alanine | 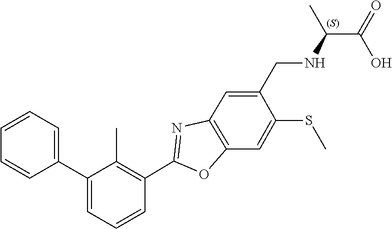 | 433.2 |
| 6 | ((2-(2-methyl-[1,1′-biphenyl]-3-yl)- 6-(methylthio)benzo[d]oxazol-5- yl)methyl)-D-alanine | 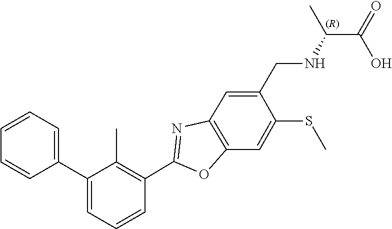 | 433.2 |
| 7 | ((2-(2-methyl-[1,1′-biphenyl]-3-yl)- 6-(methylthio)benzo[d]oxazol-5- yl)methyl)-D-proline | 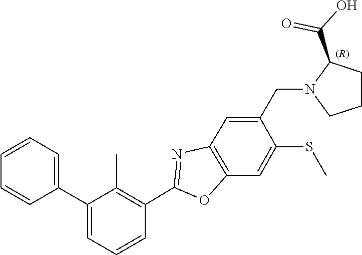 | 459.2 |
| 8 | (1-((2-(2-methyl-[1,1′-biphenyl]-3- yl)-6-(methylthio)benzo[d]oxazol- 5-yl)methyl)piperidin-2- yl)methanol | 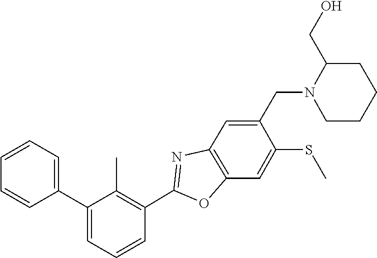 [SMILES: CSc1cc2oc(-c3cccc(-c4ccccc4)c3C)nc2cc1CN1CCCCC1CO] | 459.2 |
| 9 | (S)-4-((2-(2-methyl-[1,1′-biphenyl]- 3-yl)-6- (methylthio)benzo[d]oxazol-5- yl)methyl)morpholine-3-carboxylic acid | 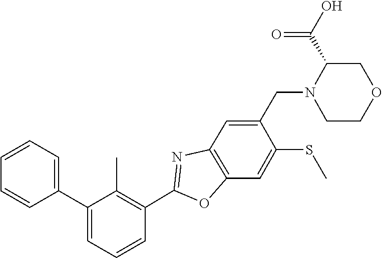 [SMILES: CSc1cc2oc(-c3cccc(-c4ccccc4)c3C)nc2cc1CN1CCOC[C@H]1C(=O)O] | 475.2 |
| 10 | (S)-2-(((2-(2-methyl-[1,1′- biphenyl]-3-yl)-6- (methylthio)benzo[d]oxazol-5- yl)methyl)amino)butanoic acid | 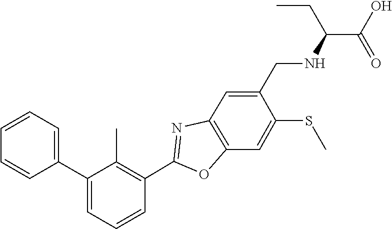 [SMILES: CC[C@H](NCc1cc2nc(-c3cccc(-c4ccccc4)c3C)oc2cc1SC)C(=O)O] | 447.2 |
| 11 | (R)-2-(((2-(2-methyl-[1,1′- biphenyl]-3-yl)-6- (methylthio)benzo[d]oxazol-5- yl)methyl)amino)butanoic acid | 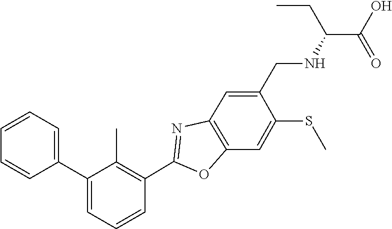 [SMILES: CC[C@@H](NCc1cc2nc(-c3cccc(-c4ccccc4)c3C)oc2cc1SC)C(=O)O] | 447.2 |
| 12 | ((2-(2-methyl-[1,1′-biphenyl]-3-yl)- 6-(methylthio)benzo[d]oxazol-5- yl)methyl)-D-serine | 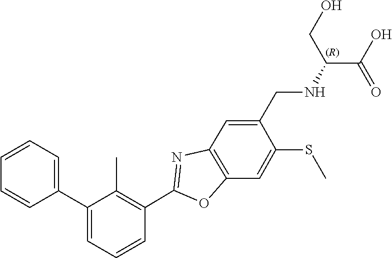 | 449.2 |
| 13 | ((2-(2-methyl-[1,1′-biphenyl]-3-yl)- 6-(methylthio)benzo[d]oxazol-5- yl)methyl)-L-serine | 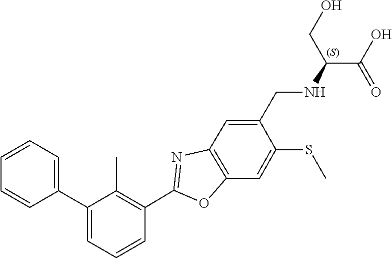 | 449.2 |
| 14 | ((2-(2-cyano-[1,1′-biphenyl]-3-yl)- 6-(methylthio)benzo[d]oxazol-5- yl)methyl)-L-proline | 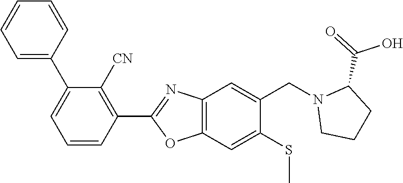 [SMILES: CSc1cc2oc(-c3cccc(-c4ccccc4)c3C#N)nc2cc1CN1CCC[C@H]1C(=O)O] | 470.2 |
| 15 | ((2-(2,2′-dicyano-[1,1′-biphenyl]-3- yl)-6-(methylthio)benzo[d]oxazol- 5-yl)methyl)-L-proline | 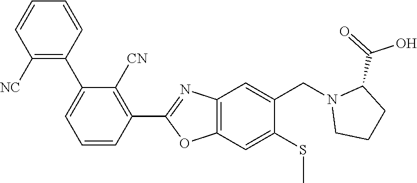 [SMILES: CSc1cc2oc(-c3cccc(-c4ccccc4C#N)c3C#N)nc2cc1CN1CCC[C@H]1C(=O)O] | 495.1 |
| 16 | 2-methyl-1-((2-(2-methyl-[1,1′- biphenyl]-3-yl)-6- (methylthio)benzo[d]oxazol-5- yl)methyl)pyrrolidine-2-carboxylic acid | 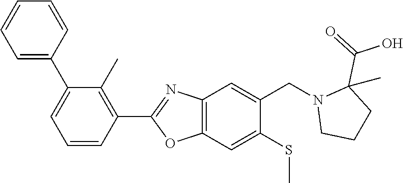 [SMILES: CSc1cc2oc(-c3cccc(-c4ccccc4)c3C)nc2cc1CN1CCCC1(C)C(=O)O] | 473.2 |
| 17 | ((2-(2-cyano-[1,1′-biphenyl]-3-yl)- 6-methylbenzo[d]oxazol-5- yl)methyl)-D-proline | 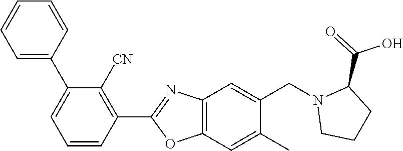 [SMILES: Cc1cc2oc(-c3cccc(-c4ccccc4)c3C#N)nc2cc1CN1CCC[C@@H]1C(=O)O] | 438.2 |
| 18 | ((2-(2-cyano-[1,1′-biphenyl]-3-yl)- 6-methylbenzo[d]oxazol-5- yl)methyl)-L-proline | 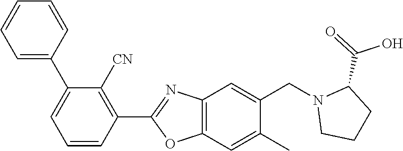 [SMILES: Cc1cc2oc(-c3cccc(-c4ccccc4)c3C#N)nc2cc1CN1CCC[C@H]1C(=O)O] | 438.2 |
| 19 | (S)-4-((2-(2-cyano-[1,1′-biphenyl]- 3-yl)-6-methylbenzo[d]oxazol-5- yl)methyl)morpholine-3-carboxylic acid | 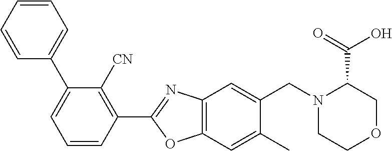 [SMILES: Cc1cc2oc(-c3cccc(-c4ccccc4)c3C#N)nc2cc1CN1CCOC[C@H]1C(=O)O] | 454.2 |
| 20 | ((6-chloro-2-(2-methyl-[1,1′- biphenyl]-3-yl)benzo[d]oxazol-5- yl)methyl)-L-proline | 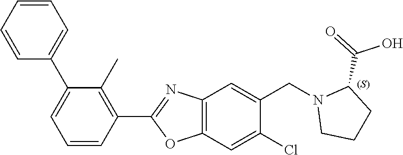 | 448.1 |
| 21 | ((6-hydroxy-2-(2-methyl-[1,1′- biphenyl]-3-yl)benzo[d]oxazol-5- yl)methyl)-L-proline | 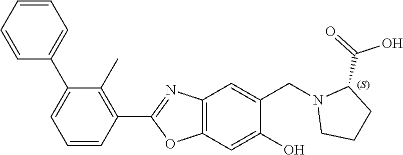 | 429.2 |
| 22 | (S)-1-((6-methyl-2-(2-methyl-[1,1′- biphenyl]-3-yl)benzo[d]oxazol-5- yl)methyl)piperidine-2-carboxylic acid | 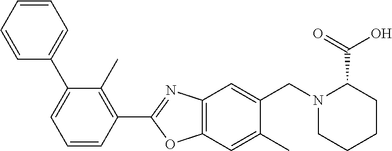 [SMILES: Cc1cc2oc(-c3cccc(-c4ccccc4)c3C)nc2cc1CN1CCCC[C@H]1C(=O)O] | 441.2 |
| 23 | (S)-1-((6-fluoro-2-(2-methyl-[1,1′- biphenyl]-3-yl)benzo[d]oxazol-5- yl)methyl)pyrrolidin-3-ol | 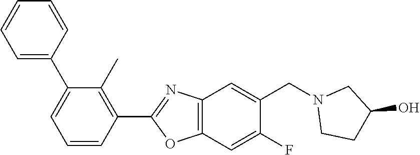 [SMILES: Cc1c(-c2ccccc2)cccc1-c1nc2cc(CN3CC[C@H](O)C3)c(F)cc2o1] | 403.2 |
| 24 | ((6-fluoro-2-(2-methyl-[1,1′- biphenyl]-3-yl)benzo[d]oxazol-5- yl)methyl)-L-proline | 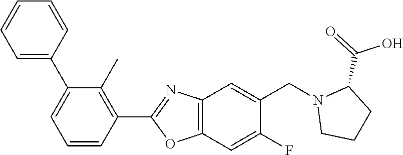 [SMILES: Cc1c(-c2ccccc2)cccc1-c1nc2cc(CN3CCC[C@H]3C(=O)O)c(F)cc2o1] | 431.2 |
| 25 | (S)-1-((6-fluoro-2-(2-methyl-[1,1′- biphenyl]-3-yl)benzo[d]oxazol-5- yl)methyl)piperidine-2-carboxylic acid | 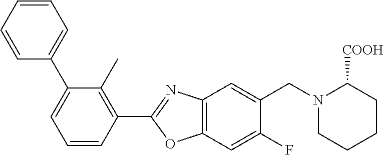 [SMILES: Cc1c(-c2ccccc2)cccc1-c1nc2cc(CN3CCCC[C@H]3C(=O)O)c(F)cc2o1] | 445.2 |
| 26 | 1-((6-cyano-2-(2-methyl-[1,1′- biphenyl]-3-yl)benzo[d]oxazol-5- yl)methyl)piperidine-2-carboxylic acid | 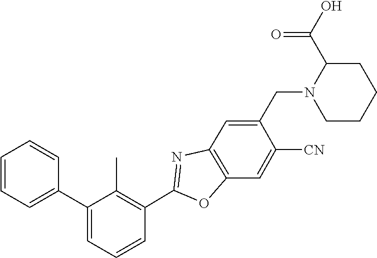 [SMILES: Cc1c(-c2ccccc2)cccc1-c1nc2cc(CN3CCCCC3C(=O)O)c(C#N)cc2o1] | 452.2 |
| 27 | 1-((2-(2-methyl-[1,1′-biphenyl]-3- yl)-6- (methylamino)benzo[d]oxazol-5- yl)methyl)piperidine-2-carboxylic acid | 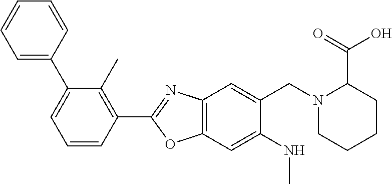 [SMILES: CNc1cc2oc(-c3cccc(-c4ccccc4)c3C)nc2cc1CN1CCCCC1C(=O)O] | 456.2 |
| 28 | ((2-(2-methyl-[1,1′-biphenyl]-3-yl)- 6-(trifluoromethyl)benzo[d]oxazol- 5-yl)methyl)-L-proline | 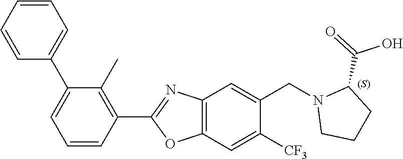 | 481.2 |

### Example 29 Synthesis of Compound 29

### (S)-1-((6-(difluoromethoxy)-2-(2-methyl-[1,1′-biphenyl]-3-yl)benzo[d]oxazol-5-yl)methyl)piperidine-2-carboxylic acid

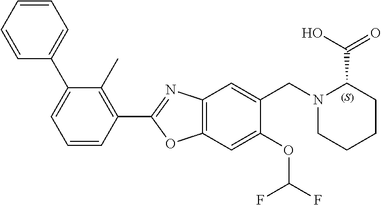

### Step 1: Preparation of methyl 2,4-dihydroxy-5-nitrobenzoate

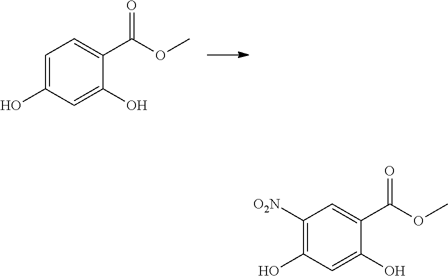

Methyl 2,4-dihydroxybenzoate (850 g, 5.06 mol) was dissolved in a mixture of AcOH (3.6 L) and Ac₂O (900 mL). After cooling the clarified solution to 10° C. (ice bath), a mixture of HNO₃ (65%) (455 ml) in AcOH (500 mL) was added over 1 h. When the addition was completed, rose the temperature of the mixture solution to 15-20° C. and stirring for another 1 h. Until the raw material was almost finished and the reaction stopped. Poured the reaction solution into H₂O (3 L), then the mixture was added for another 30 min without stirring. The precipitate was filtered, rinsed with small amounts of H₂O. Then poured the crude product into MeOH (2 L) with stirring. The precipitate was filtered, rinsed with small amounts of MeOH. Dried under vacuum to get the product 480 g methyl 2,4-dihydroxy-5-nitrobenzoate.

### Step 2: Preparation of methyl 5-amino-2,4-dihydroxybenzoate

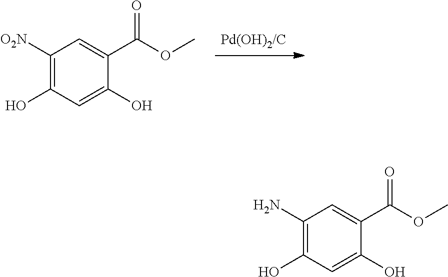

A mixture of methyl 2,4-dihydroxy-5-nitrobenzoate (77.1 g) and 10% Pd/C (11.5 g) in methanol (2 L) was stirred under 1.1 atm of hydrogen pressure at room temperature for 3 h. The catalyst was removed by filtration, the solid residue was washed with methanol (300 mL) and then dried under vacuum to obtain 72 g crude product methyl 5-amino-2,4-dihydroxybenzoate.

### Step 3: Preparation of methyl 2-(3-bromo-2-methylphenyl)-6-hydroxybenzo[d]oxazole-5-carboxylate

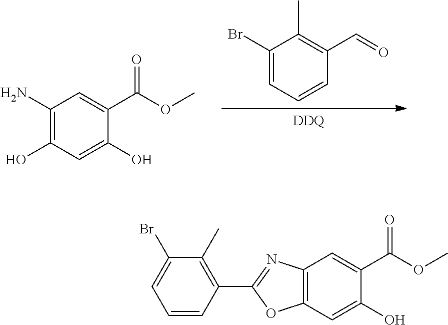

A mixture of methyl 5-amino-2,4-dihydroxybenzoate (32.9 g) and 3-bromo-2-methylbenzaldehyde (32.5 g) in MeOH (1 L) was stirred for 2.5 h at 80° C., then the mixture was concentered under reduced pressure. The mixture was added DCM (500 ml) and DDQ (55.6 g). The mixture was stirred at room temperature for 1 h. The reaction was diluted with DCM and washed with an aqueous Na₂S₂O₃ solution and NaHCO₃ solution. The organic phase was dried over MgSO₄, filtered and the filtrate was concentrated. The crude product was purified by column chromatography (PE:DCM=1:1) to afford 45 g methyl 2-(3-bromo-2-methylphenyl)-6-hydroxybenzo[d]oxazole-5-carboxylate as a brown solid.

### Step 4: Preparation of methyl 2-(3-bromo-2-methylphenyl)-6-(difluoromethoxy)benzo[d]oxazole-5-carboxylate

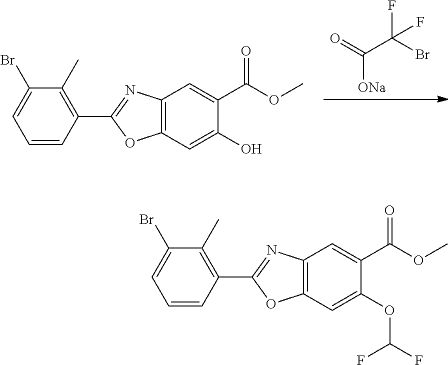

Methyl 2-(3-bromo-2-methylphenyl)-6-hydroxybenzo[d]oxazole-5-carboxylate (10.0 g), sodium 2-bromo-2,2-difluoroacetate (5.46 g), Cs₂CO₃(27.09 g), KI (4.59 g), TBAI (10.22 g) was dissolved in DMF (200 mL), The mixture was stirred at 100° C. for 3 h, The reaction was diluted with DCM and washed with a saturated NaCl solution, The crude product was purified by column chromatography (PE:DCM=1:1) to afford 5 g methyl 2-(3-bromo-2-methylphenyl)-6-(difluoromethoxy)benzo[d]oxazole-5-carboxylate.

### Step 5: Preparation of methyl 6-(difluoromethoxy)-2-(2-methyl-[1,1′-biphenyl]-3-yl)benzo[d]oxazole-5-carboxylate

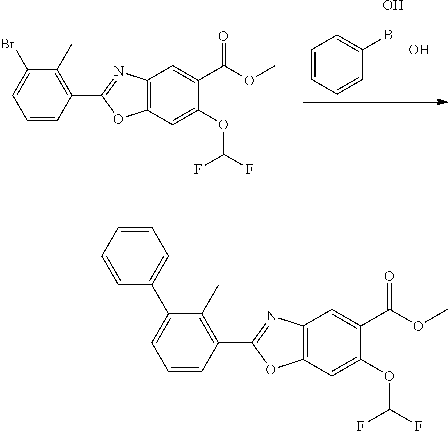

Methyl 2-(3-bromo-2-methylphenyl)-6-(difluoromethoxy)benzo[d]oxazole-5-carboxylate (6.00 g), phenylboronic acid (2.70 g), K₂CO₃(6.10 g), Pd(dppf)Cl₂(1.10 g) was added to dioxiane (50 ml) under nitrogen at 80° C. for 120 min. The mixture was cooled and diluted with DCM, then washed with H₂O and NaCl solution. The organic phase was dried over MgSO₄, filtered and the filtrate was concentrated. The crude product was purified by column chromatography (PE:DCM=1:5) to afford 5 g methyl 6-(difluoromethoxy)-2-(2-methyl-[1,1′-biphenyl]-3-yl)benzo[d]oxazole-5-carboxylate.

### Step 6: Preparation of (6-(difluoromethoxy)-2-(2-methyl-[1,1′-biphenyl]-3-yl)benzo[d]oxazol-5-yl)methanol

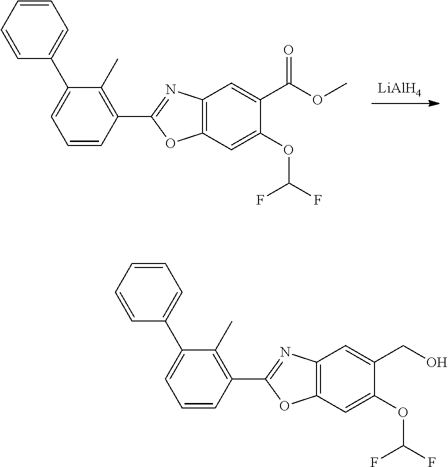 [SMILES: COC(=O)c1cc2nc(-c3cccc(-c4ccccc4)c3C)oc2cc1OC(F)F ; "LiAlH4" ; Cc1c(-c2ccccc2)cccc1-c1nc2cc(CO)c(OC(F)F)cc2o1]

To a solution of methyl 6-(difluoromethoxy)-2-(2-methyl-[1,1′-biphenyl]-3-yl)benzo[d]oxazole-5-carboxylate (1.30 g) and THF (50 mL) was added LiAlH4 in THF (2.5 M, 5 mL) dropwise at 0° C., The mixture was warmed up to room temperature. After 1 h, the mixture was quenched with 1 mL H₂O and 1 mL 10% NaOH solution, then washed with 1 M HCl, water and brine separately. The organic phase was dried over Na₂SO₄, filtered and concentrated the filtrate to obtain (6-(difluoromethoxy)-2-(2-methyl-[1,1′-biphenyl]-3-yl)benzo[d]oxazol-5-yl)methanol as a yellow solid (1.2 g).

### Step 7: Preparation of 6-(difluoromethoxy)-2-(2-methyl-[1,1′-biphenyl]-3-yl)benzo[d]oxazole-5-carbaldehyde

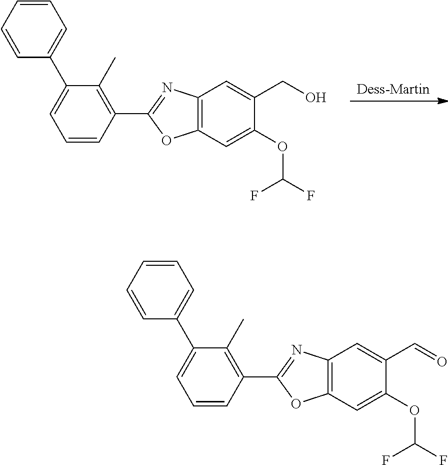 [SMILES: Cc1c(-c2ccccc2)cccc1-c1nc2cc(CO)c(OC(F)F)cc2o1 ; "Dess-Martin" ; Cc1c(-c2ccccc2)cccc1-c1nc2cc(C=O)c(OC(F)F)cc2o1]

To a solution of (6-(difluoromethoxy)-2-(2-methyl-[1,1′-biphenyl]-3-yl)benzo[d]oxazol-5-yl)methanol (1.40 g) in dry THF (15 mL) was added Dess-Martin (2.39 g) at 10° C. Then the mixture solution was stirred for 1 h at room temperature. The mixture was filtered through Celite. The solid was washed with DCM, and the combined filtrate was washed with NaHCO₃ aqueous solution, water and brine separately, dried and concentrated. The residue was purified by column chromatography (eluting with hexane-EtOAc using a gradient from 20:1 to 5:1) to afford 6-(difluoromethoxy)-2-(2-methyl-[1,1′-biphenyl]-3-yl)benzo[d]oxazole-5-carbaldehyde as solid (1.27 g).

### Step 8: Preparation of (S)-1-((6-(difluoromethoxy)-2-(2-methyl-[1,1′-biphenyl]-3-yl)benzo[d]oxazol-5-yl)methyl)piperidine-2-carboxylic acid

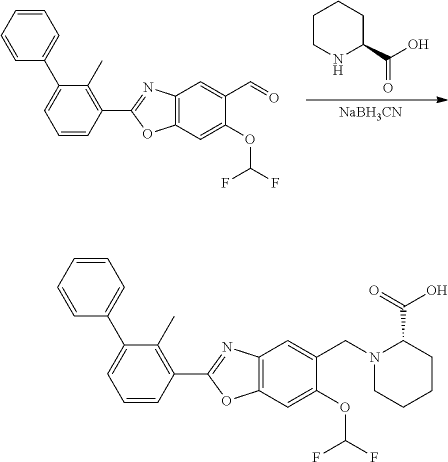

A mixture of 6-(difluoromethoxy)-2-(2-methyl-[1,1′-biphenyl]-3-yl)benzo[d]oxazole-5-carbaldehyde (1.00 g), (S)-piperidine-2-carboxylic acid (1.70 g), AcOH (316 mg) in MeOH was stirred at room temperature for 0.5 hrs. Then NaBH₃CN (498 mg) was added, heated at 60° C. for 2 h. The mixture was cooled and diluted with DCM and then washed with H₂O and NaCl solution separately. The organic phase was dried over MgSO₄, filtered and concentrated the filtrate. The residue was purified by column chromatography (DCM:MeOH=8%), to afford (S)-1-((6-(difluoromethoxy)-2-(2-methyl-[1,1′-biphenyl]-3-yl)benzo[d]oxazol-5-yl)methyl)piperidine-2-carboxylic acid as a white solid. (671 mg).

### Example 30 Synthesis of Compound 30

### ((6-(difluoromethoxy)-2-(2-methyl-[1,1′-biphenyl]-3-yl)benzo[d]oxazol-5-yl)methyl)-D-allothreonine

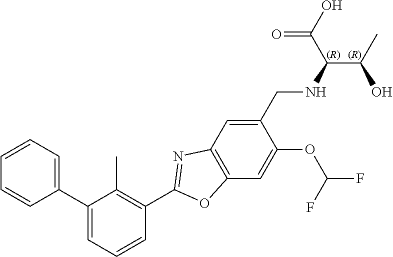

Prepare the Compound 30 essentially as described for Example 29 using the corresponding intermediate. For example, using

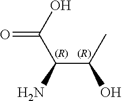

instead of

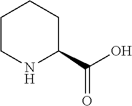

in the last step (step 8) described above.

### Example 36 Synthesis of Compound 36

### ((6-(difluoromethoxy)-2-(2-methyl-[1,1′-biphenyl]-3-yl)benzo[d]oxazol-5-yl)methyl)-L-proline

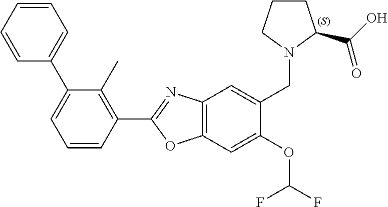

Prepare the Compound 36 essentially as described for Example 29 using the corresponding intermediate. For example, using “L-proline” instead of “(S)-piperidine-2-carboxylic acid” in the last step (step 8) described above. The example procedure of preparing Compound 29 was described as follows:

A solution of 6-(difluoromethoxy)-2-(2-methyl-[1,1′-biphenyl]-3-yl)benzo[d]oxazole-5-carbaldehyde (1.00 g), L-proline (1.60 g), HOAC (325 mg) in MeOH was stirred at room temperature for 0.5 hrs. The mixture was added NaBH₃CN (498 mg), then was heated at 60° C. for 2 h. The mixture was cooled and diluted with DCM and washed with H₂O and NaCl solution. The organic phase was dried over MgSO₄, filtered and the filtrate was concentrated. The residue was purified by column chromatography (DCM:MeOH=8%) to afford ((6-(difluoromethoxy)-2-(2-methyl-[1,1′-biphenyl]-3-yl)benzo[d]oxazol-5-yl)methyl)-L-proline as a white solid (650 mg).

Prepare the following examples (shown in Table 2) essentially as described for Example 29 using the corresponding intermediates, i.e. amino acid, for example L-alanine, glycine, L-proline instead of (S)-piperidine-2-carboxylic acid in the step 8 described above.

TABLE 2

| | | | Physical |
|---|---|---|---|
| EX | | | Data (MS) |
| No. | Chemical Name | Structure | (M + H)⁺ |
| 29 | (S)-1-((6-(difluoromethoxy)-2-(2- methyl-[1,1′-biphenyl]-3- yl)benzo[d]oxazol-5- yl)methyl)piperidine-2-carboxylic acid | 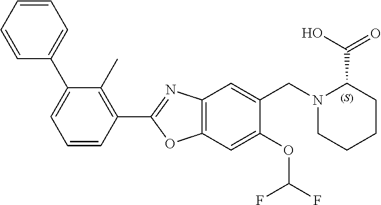 | 493.2 |
| 30 | ((6-(difluoromethoxy)-2-(2-methyl- [1,1′-biphenyl]-3- yl)benzo[d]oxazol-5-yl)methyl)-D- allothreonine | 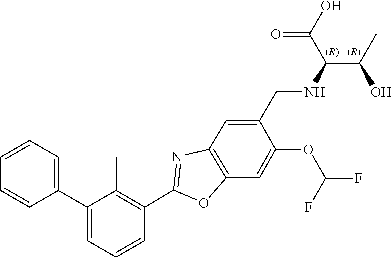 | 483.2 |
| 31 | ((6-(difluoromethoxy)-2-(2-methyl- [1,1′-biphenyl]-3- yl)benzo[d]oxazol-5-yl)methyl)-L- allothreonine | 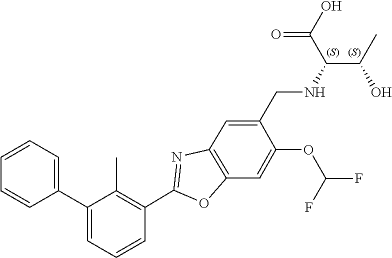 | 483.2 |
| 32 | ((6-(difluoromethoxy)-2-(2-methyl- [1,1′-biphenyl]-3- yl)benzo[d]oxazol-5-yl)methyl)-L- tyrosine hydrochloride | 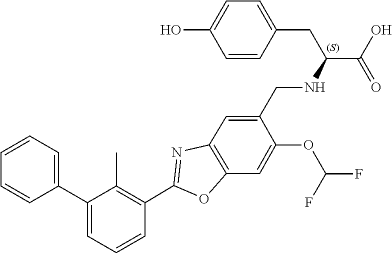 | 581.2 |
| 3 | ((6-(difluoromethoxy)-2-(2-methyl- [1,1′-biphenyl]-3- yl)benzo[d]oxazol-5-yl)methyl)-L- alanine | 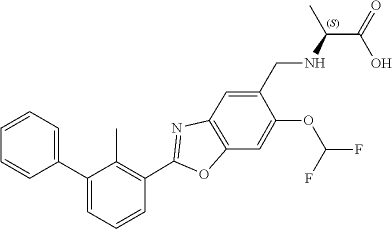 | 453.2 |
| 34 | ((6-(difluoromethoxy)-2-(2-methyl- [1,1′-biphenyl]-3- yl)benzo[d]oxazol-5-yl)methyl)-D- alanine | 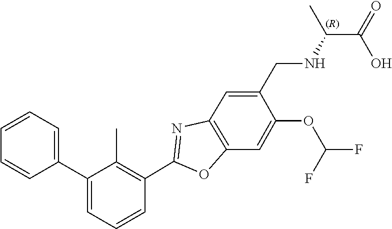 | 453.2 |
| 35 | ((6-(difluoromethoxy)-2-(2-methyl- [1,1′-biphenyl]-3- yl)benzo[d]oxazol-5- yl)methyl)glycine | 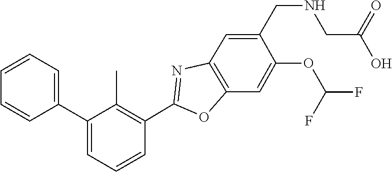 [SMILES: Cc1c(-c2ccccc2)cccc1-c1nc2cc(CNCC(=O)O)c(OC(F)F)cc2o1] | 439.1 |
| 36 | ((6-(difluoromethoxy)-2-(2-methyl- [1,1′-biphenyl]-3- yl)benzo[d]oxazol-5-yl)methyl)-L- proline | 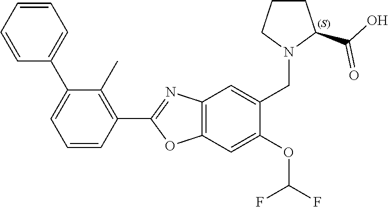 | 479.2 |
| 37 | ((6-(difluoromethoxy)-2-(2-methyl- [1,1′-biphenyl]-3- yl)benzo[d]oxazol-5-yl)methyl)-D- proline | 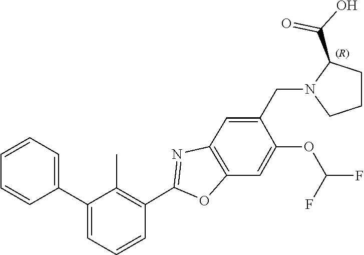 | 479.2 |
| 38 | (1-((6-(difluoromethoxy)-2-(2- methyl-[1,1′-biphenyl]-3- yl)benzo[d]oxazol-5- yl)methyl)piperidin-2-yl)methanol | 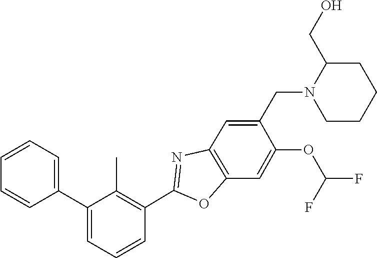 [SMILES: Cc1c(-c2ccccc2)cccc1-c1nc2cc(CN3CCCCC3CO)c(OC(F)F)cc2o1] | 479.2 |
| 39 | (S)-4-((6-(difluoromethoxy)-2-(2- methyl-[1,1′-biphenyl]-3- yl)benzo[d]oxazol-5- yl)methyl)morpholine-3-carboxylic acid | 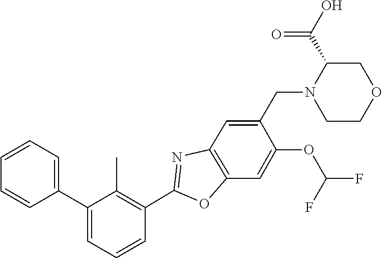 [SMILES: Cc1c(-c2ccccc2)cccc1-c1nc2cc(CN3CCOC[C@H]3C(=O)O)c(OC(F)F)cc2o1] | 495.2 |
| 40 | (S)-2-(((6-(difluoromethoxy)-2-(2- methyl-[1,1′-biphenyl]-3- yl)benzo[d]oxazol-5- yl)methyl)amino)butanoic acid | 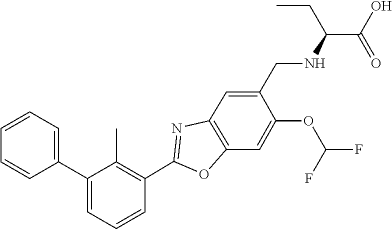 [SMILES: CC[C@H](NCc1cc2nc(-c3cccc(-c4ccccc4)c3C)oc2cc1OC(F)F)C(=O)O] | 467.2 |
| 41 | (R)-2-(((6-(difluoromethoxy)-2-(2- methyl-[1,1′-biphenyl]-3- yl)benzo[d]oxazol-5- yl)methyl)amino)butanoic acid | 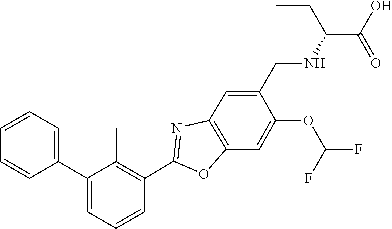 [SMILES: CC[C@@H](NCc1cc2nc(-c3cccc(-c4ccccc4)c3C)oc2cc1OC(F)F)C(=O)O] | 467.2 |
| 42 | ((6-(difluoromethoxy)-2-(2-methyl- [1,1′-biphenyl]-3- yl)benzo[d]oxazol-5-yl)methyl)-D- serine | 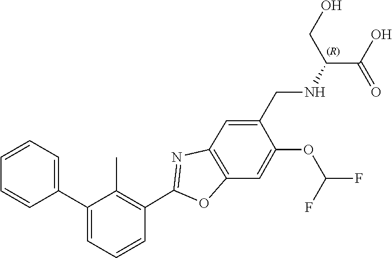 | 469.2 |
| 43 | ((6-(difluoromethoxy)-2-(2-methyl- [1,1′-biphenyl]-3- yl)benzo[d]oxazol-5-yl)methyl)-L- serine | 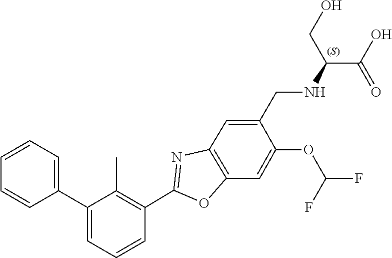 | 469.2 |
| 44 | ((2-(2-cyano-[1,1′-biphenyl]- 3-yl)-6- (difluoromethoxy)benzo[d]oxazol- 5-yl)methyl)-L-proline | 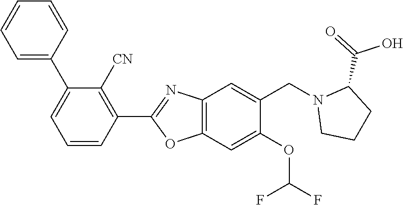 [SMILES: N#Cc1c(-c2ccccc2)cccc1-c1nc2cc(CN3CCC[C@H]3C(=O)O)c(OC(F)F)cc2o1] | 490.2 |
| 45 | ((2-(2,2′-dicyano-[1,1′-biphenyl]-3- yl)-6- (difluoromethoxy)benzo[d]oxazol- 5-yl)methyl)-L-proline | 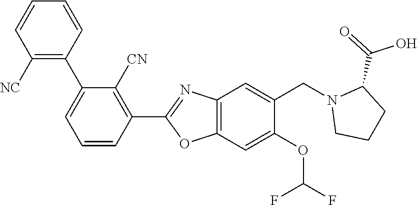 [SMILES: N#Cc1ccccc1-c1cccc(-c2nc3cc(CN4CCC[C@H]4C(=O)O)c(OC(F)F)cc3o2)c1C#N] | 515.2 |
| 46 | 1-((6-(difluoromethoxy)-2-(2- methyl-[1,1′-biphenyl]-3- yl)benzo[d]oxazol-5-yl)methyl)-2- methylpyrrolidine-2-carboxylic acid | 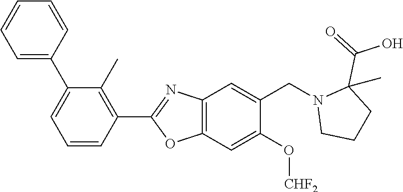 [SMILES: Cc1c(-c2ccccc2)cccc1-c1nc2cc(CN3CCCC3(C)C(=O)O)c(OC(F)F)cc2o1] | 493.2 |
| 47 | ((6-(difluoromethoxy)-2-(3-(2,3- dihydrobenzo[b][1,4]dioxin-6-yl)- 2-methylphenyl)benzo[d]oxazol-5- yl)methyl)-L-proline | 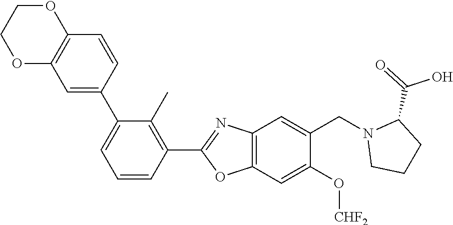 [SMILES: Cc1c(-c2ccc3c(c2)OCCO3)cccc1-c1nc2cc(CN3CCC[C@H]3C(=O)O)c(OC(F)F)cc2o1] | 537.2 |
| 48 | 1-((6-(difluoromethoxy)-2-(2- methyl-[1,1′-biphenyl]-3- yl)benzo[d]oxazol-5- yl)methyl)azetidine-2-carboxylic acid | 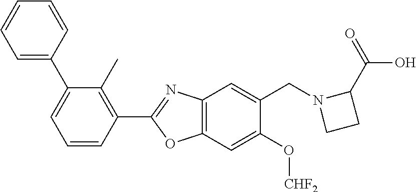 [SMILES: Cc1c(-c2ccccc2)cccc1-c1nc2cc(CN3CCC3C(=O)O)c(OC(F)F)cc2o1] | 465.2 |
| 49 | ((6-(difluoromethoxy)-2-(3-(2,3- dihydrobenzo[b][1,4]dioxin-6-yl)- 2-methylphenyl)benzo[d]oxazol-5- yl)methyl)-L-proline | 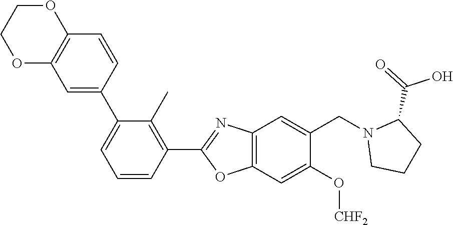 [SMILES: Cc1c(-c2ccc3c(c2)OCCO3)cccc1-c1nc2cc(CN3CCC[C@H]3C(=O)O)c(OC(F)F)cc2o1] | 537.2 |
| 50 | 1-((6-(difluoromethoxy)-2-(2- methyl-[1,1′-biphenyl]-3- yl)benzo[d]oxazol-5-yl)methyl)-2- methylpyrrolidine-2-carboxylic acid | 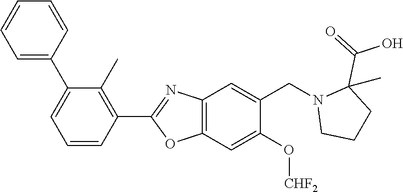 [SMILES: Cc1c(-c2ccccc2)cccc1-c1nc2cc(CN3CCCC3(C)C(=O)O)c(OC(F)F)cc2o1] | 493.2 |
| 51 | ((2-(2′-cyano-2-methyl-[1,1′- biphenyl]-3-yl)-6- (difluoromethoxy)benzo[d]oxazol- 5-yl)methyl)proline | 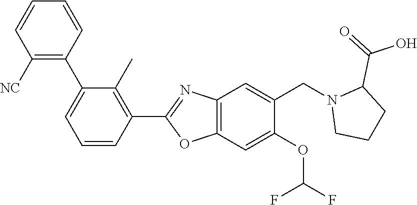 [SMILES: Cc1c(-c2nc3cc(CN4CCCC4C(=O)O)c(OC(F)F)cc3o2)cccc1-c1ccccc1C#N] | 504.2 |
| 52 | ((6-(difluoromethoxy)-2-(2′-fluoro- 2-methyl-[1,1′-biphenyl]-3- yl)benzo[d]oxazol-5-yl)methyl)-L- proline | 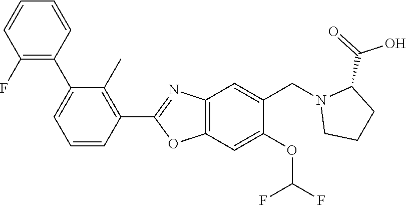 [SMILES: Cc1c(-c2nc3cc(CN4CCC[C@H]4C(=O)O)c(OC(F)F)cc3o2)cccc1-c1ccccc1F] | 497.2 |
| 53 | (S)-3((6-(difluoromethoxy)-2-(2- methyl-[1,1′-biphenyl]-3- yl)benzo[d]oxazol-5- yl)methyl)oxazolidine-4-carboxylic acid | 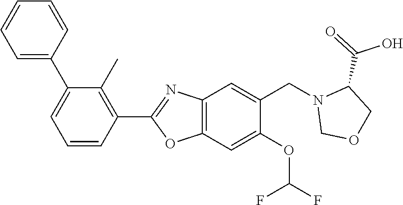 [SMILES: Cc1c(-c2ccccc2)cccc1-c1nc2cc(CN3COC[C@H]3C(=O)O)c(OC(F)F)cc2o1] | 481.2 |

### Example 54 Synthesis of Compound 54

### (S)-1-((2-(2-methyl-[1,1′-biphenyl]-3-yl)-6-(2, 2,2-trifluoroethoxy)benzo[d]oxazol-5-yl)methyl)piperidine-2-carboxylic acid

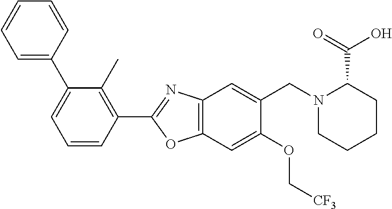 [SMILES: Cc1c(-c2ccccc2)cccc1-c1nc2cc(CN3CCCC[C@H]3C(=O)O)c(OCC(F)(F)F)cc2o1]

Prepare the Compound 54 essentially as described for Example 1 using the corresponding intermediate, for example, using

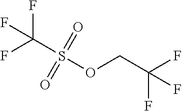 [SMILES: O=S(=O)(OCC(F)(F)F)C(F)(F)F]

instead of

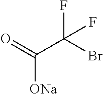

in the step 4 described above. The example procedure of preparing methyl 2-(3-bromo-2-methylphenyl)-6-(2,2,2-trifluoroethoxy)benzo[d]oxazole-5-carboxylate was described as follows:

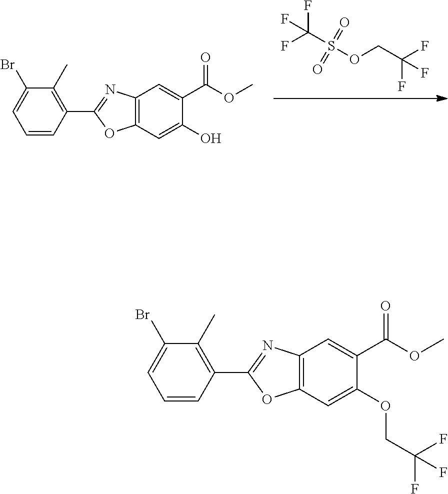 [SMILES: COC(=O)c1cc2nc(-c3cccc(Br)c3C)oc2cc1O ; O=S(=O)(OCC(F)(F)F)C(F)(F)F ; COC(=O)c1cc2nc(-c3cccc(Br)c3C)oc2cc1OCC(F)(F)F]

To a refluxing mixture of acetone (20 mL), methyl 2-(3-bromo-2-methylphenyl)-6-hydroxybenzo[d]oxazole-5-carboxylate (2.0 g) and potassium carbonate (3.1 g) in a 40 mL sealed vial was added dropwise 2,2,2-trifluoroethyl trifluoromethanesulfonate (3.9 g). The mixture was stirred at reflux overnight and then concentrated under reduced pressure. The residue was dissolved in water (50 mL) and dichloromethane (50 mL). The layers were separated and the aqueous layer was extracted with dichloromethane (3*50 mL). The combined organic extracts were dried (Na₂SO₄), filtered and concentrated to afford crude methyl 2-(3-bromo-2-methylphenyl)-6-(2,2,2-trifluoroethoxy)benzo[d]oxazole-5-carboxylate 1.8 g.

Prepare the following examples (shown in Table 3) essentially as described for Example 54 using the corresponding intermediates, i.e. amino acid, for example L-alanine, glycine, L-proline instead of (S)-piperidine-2-carboxylic acid.

TABLE 3

| | | | Physical |
|---|---|---|---|
| EX | | | Data (MS) |
| No. | Chemical Name | Structure | (M + H)⁺ |
| 54 | (S)-1-((2-(2-methyl-[1,1′- biphenyl]-3-yl)-6-(2,2,2- trifluoroethoxy)benzo[d]oxazol-5- yl)methyl)piperidine-2-carboxylic acid | 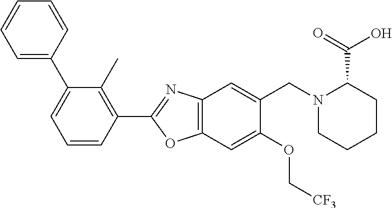 [SMILES: Cc1c(-c2ccccc2)cccc1-c1nc2cc(CN3CCCC[C@H]3C(=O)O)c(OCC(F)(F)F)cc2o1] | 525.2 |
| 55 | 1-((6-(2,2-difluoroethoxy)-2-(2- methyl-[1,1′-biphenyl]-3- yl)benzo[d]oxazol-5- yl)methyl)piperidine-2-carboxylic acid | 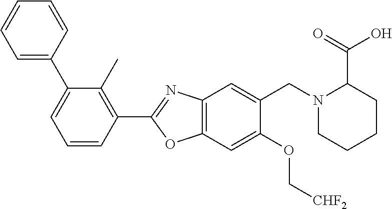 [SMILES: Cc1c(-c2ccccc2)cccc1-c1nc2cc(CN3CCCCC3C(=O)O)c(OCC(F)F)cc2o1] | 507.2 |
| 56 | ((2-(2-methyl-[1,1′-biphenyl]-3-yl)- 6-(2,2,2- trifluoroethoxy)benzo[d]oxazol-5- yl)methyl)-D-alanine | 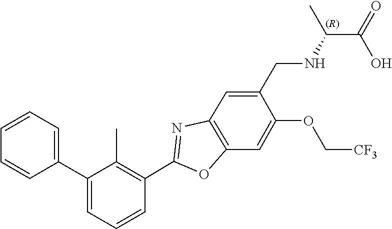 | 485.2 |
| 57 | (S)-4-((2-(2-cyano-[1,1′-biphenyl]- 3-yl)-6-(2,2,2- trifluoroethoxy)benzo[d]oxazol-5- yl)methyl)morpholine-3-carboxylic acid | 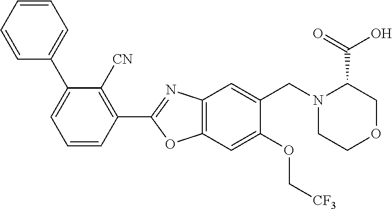 [SMILES: N#Cc1c(-c2ccccc2)cccc1-c1nc2cc(CN3CCOC[C@H]3C(=O)O)c(OCC(F)(F)F)cc2o1] | 538.2 |
| 58 | (S)-4-((2-(2-methyl-[1,1′- biphenyl]-3-yl)-6-(2,2,2- trifluoroethoxy)benzo[d]oxazol-5- yl)methyl)morpholine-3-carboxylic acid | 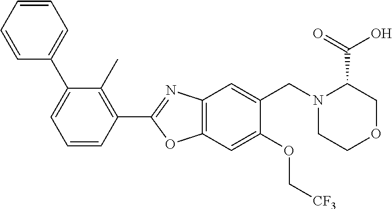 [SMILES: Cc1c(-c2ccccc2)cccc1-c1nc2cc(CN3CCOC[C@H]3C(=O)O)c(OCC(F)(F)F)cc2o1] | 527.2 |
| 59 | ((2-(2-methyl-[1,1′-biphenyl]-3-yl)- 6-(2,2,2- trifluoroethoxy)benzo[d]oxazol-5- yl)methyl)-L-alanine | 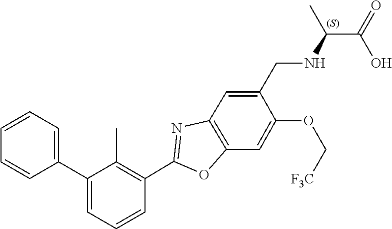 | 485.2 |
| 60 | ((2-(2-methyl-[1,1′-biphenyl]-3-yl)- 6-(2,2,2- trifluoroethoxy)benzo[d]oxazol-5- yl)methyl)-L-proline | 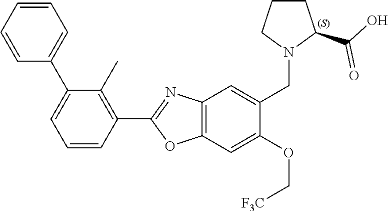 | 511.2 |
| 61 | 2-methyl-1-((2-(2-methyl-[1,1′- biphenyl]-3-yl)-6-(2,2,2- trifluoroethoxy)benzo[d]oxazol-5- yl)methyl)pyrrolidine-2-carboxylic acid | 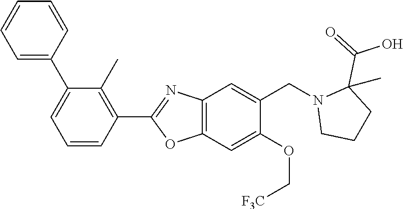 [SMILES: Cc1c(-c2ccccc2)cccc1-c1nc2cc(CN3CCCC3(C)C(=O)O)c(OCC(F)(F)F)cc2o1] | 525.2 |
| 62 | ((2-(2-methyl-[1,1′-biphenyl]-3-yl)- 6-(2,2,2- trifluoroethoxy)benzo[d]oxazol-5- yl)methyl)-D-alanine | 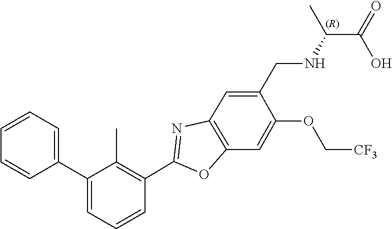 | 485.2 |
| 63 | ((2-(2-methyl-[1,1′-biphenyl]- 3-yl)-6- (trifluoromethoxy)benzo[d]oxazol- 5-yl)methyl)-L-proline | 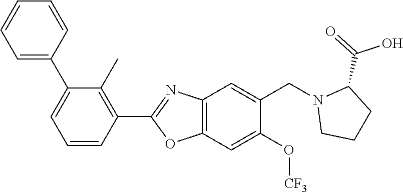 [SMILES: Cc1c(-c2ccccc2)cccc1-c1nc2cc(CN3CCC[C@H]3C(=O)O)c(OC(F)(F)F)cc2o1] | 497.2 |

### Example 64 Synthesis of Compound 64

### ((2-(2-methyl-[1,1′-biphenyl]-3-yl)-6-((methylsulfonyl)oxy)benzo[d]oxazol-5-yl)methyl)-L-proline

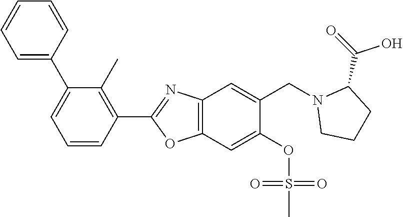 [SMILES: Cc1c(-c2ccccc2)cccc1-c1nc2cc(CN3CCC[C@H]3C(=O)O)c(OS(C)(=O)=O)cc2o1]

Prepare the Compound 64 essentially as described for Example 29 using the corresponding intermediate, for example, using “L-proline” instead of “(S)-piperidine-2-carboxylic acid” in the last step (step 8); and using

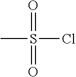

instead of

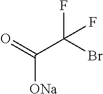

in the step 4 described above. The example procedure of preparing methyl 2-(3-bromo-2-methylphenyl)-6-((methylsulfonyl)oxy)benzo[d]oxazole-5-carboxylate was described as follows:

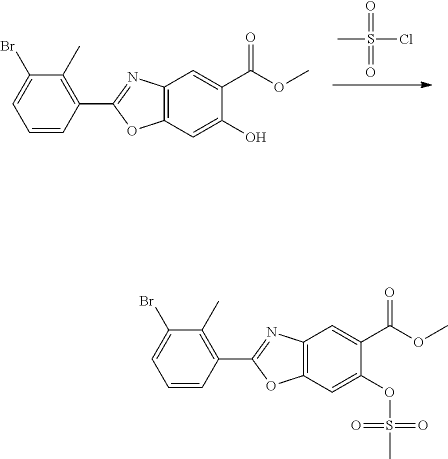

To a stirred solution of methyl 2-(3-bromo-2-methylphenyl)-6-hydroxybenzo[d]oxazole-5-carboxylate (1 g) in 30 mL of Methylene chloride was charged triethyl amine (1.2 gm). The mixture is cooled to −5 to 0° C. and methane sulfonyl chloride (1.13 gM) in Methylene chloride (10 ml) is added at 0-5° C. over a period of 1 hour maintained at 0° C. for 1 hour and raised to 22-25° C. and maintained for 3 hours. TLC showed the conversion >98%, water 20 mL is added and stirred at 22-25° C. for 1 hour. The organic layer is separated and the aqueous layer is extracted with 20 mL of Methylene chloride. The combined organic layer were washed with 20 mL of water and the solvent is evaporated to give 1.2 g of methyl 2-(3-bromo-2-methylphenyl)-6-((methylsulfonyl)oxy)benzo[d]oxazole-5-carboxylate.

### Example 65 Synthesis of Compound 65

### Step 1: Preparation of 2-(2-methyl-[1,1′-biphenyl]-3-yl)-6-(methylsulfinyl)benzo[d]oxazole-5-carbaldehyde

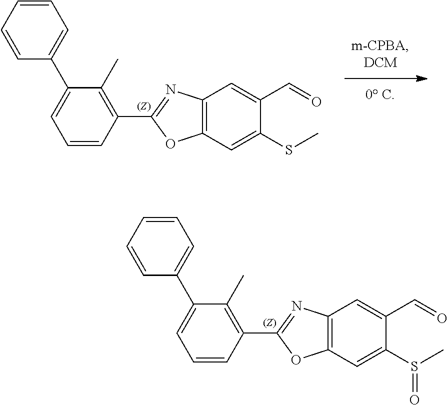 [SMILES: CSc1cc2oc(-c3cccc(-c4ccccc4)c3C)nc2cc1C=O ; Cc1c(-c2ccccc2)cccc1-c1nc2cc(C=O)c(S(C)=O)cc2o1]

The preparation of 2-(2-methyl-[1,1′-biphenyl]-3-yl)-6-(methylthio)benzo[d]oxazole-5-carbaldehyde was described as the step 6 in Example 1.

To a solution of 2-(2-methyl-[1,1′-biphenyl]-3-yl)-6-(methylthio)benzo[d]oxazole-5-carbaldehyde (700 mg) in dry DCM (15 mL) was addedm-CPBA (336 mg) at 0° C. Then the mixture solution was stirred for 0.5 h at 0° C. The mixture was filtered through Celite. The solid was washed with DCM, and the combined filtrate was washed with NaHCO₃ aqueous solution, water and brine separately, dried and concentrated. The residue was purified by column chromatography (eluting with hexane-EtOAc using a gradient from 20:1 to 5:1) to afford 2-(2-methyl-[1,1′-biphenyl]-3-yl)-6-(methylsulfinyl)benzo[d]oxazole-5-carbaldehyde as solid (1.27 g).

### Step 2: Preparation of ((2-(2-methyl-[1,1′-biphenyl]-3-yl)-6-(methylsulfinyl)benzo[d]oxazol-5-yl)methyl)-L-proline

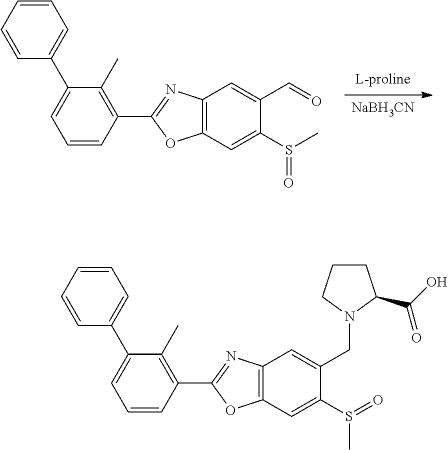 [SMILES: Cc1c(-c2ccccc2)cccc1-c1nc2cc(C=O)c(S(C)=O)cc2o1 ; "NaBH3CN" ; Cc1c(-c2ccccc2)cccc1-c1nc2cc(CN3CCC[C@H]3C(=O)O)c(S(C)=O)cc2o1]

A mixture of 2-(2-methyl-[1,1′-biphenyl]-3-yl)-6-(methylsulfinyl)benzo[d]oxazole-5-carbaldehyde, L-proline (1.70 g), HOAC (316 mg) in MeOH was stirred at room temperature for 0.5 hrs. Then NaBH₃CN (498 mg) was added, heated at 60° C. for 2 h. The mixture was cooled and diluted with DCM and then washed with H₂O and NaCl solution separately. The organic phase was dried over MgSO₄, filtered and concentrated the filtrate. The residue was purified by column chromatography (DCM:MeOH=8%), to afford ((2-(2-methyl-[1,1′-biphenyl]-3-yl)-6-(methylsulfinyl)benzo[d]oxazol-5-yl)methyl)-L-proline as a white solid (671 mg).

### Example 66 Synthesis of Compound 66

### ((2-(2-methyl-[1,1′-biphenyl]-3-yl)-6-(methylsulfonyl)benzo[d]oxazol-5-yl)methyl)-L-proline

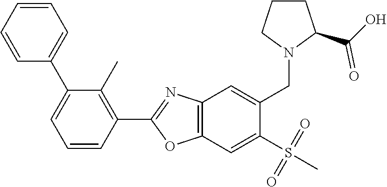 [SMILES: Cc1c(-c2ccccc2)cccc1-c1nc2cc(CN3CCC[C@H]3C(=O)O)c(S(C)(=O)=O)cc2o1]

Prepare the Compound 66 essentially as described for Example 65 using the corresponding intermediate.

### Example 67 Synthesis of Compound 67

### 8-methyl-2-(2-methyl-[1,1′-biphenyl]-3-yl)-6,7,8,9-tetrahydrooxazolo[5′,4′:4,5]benzo[1,2-f][1,4]oxazepine

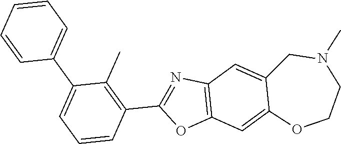 [SMILES: Cc1c(-c2ccccc2)cccc1-c1nc2cc3c(cc2o1)OCCN(C)C3]

### Step 1: Preparation of 5-(hydroxymethyl)-2-(2-methyl-[1,1′-biphenyl]-3-yl)benzo[d]oxazol-6-ol

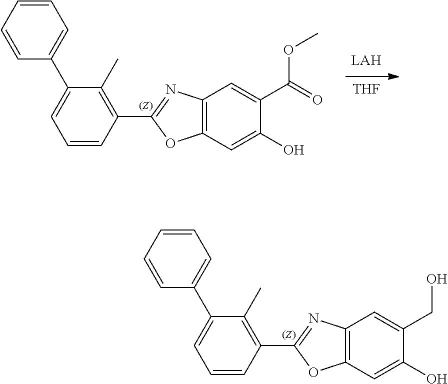 [SMILES: COC(=O)c1cc2nc(-c3cccc(-c4ccccc4)c3C)oc2cc1O ; Cc1c(-c2ccccc2)cccc1-c1nc2cc(CO)c(O)cc2o1 ; "THF"]

To methyl 6-hydroxy-2-(2-methyl-[1,1′-biphenyl]-3-yl)benzo[d]oxazole-5-carboxylate (0.5 g) was added THF (15 mL) under N2 protection, cooling to 0° C., slowly added dropwise a solution of LiAlH4 (2.5 M) in THF (0.56 mL), after the reaction completed, added 2 mL EA to quench, then added saturated NH₄Cl solution (10 mL) and EA (10 mL) for extraction. Washed with 20 mL NaCl solution twice. The organic phase was dried over Na₂SO₄, filtered and the filtrate was concentrated to obtain 0.42 g 5-(hydroxymethyl)-2-(2-methyl-[1,1′-biphenyl]-3-yl)benzo[d]oxazol-6-ol as a white solid. LCMS: [M+H]=332.0.

### Step 2: Preparation of I-(6-hydroxy-2-(2-methyl-[1,1′-biphenyl]-3-yl)benzo[d]oxazol-5-yl)ethan-1-one

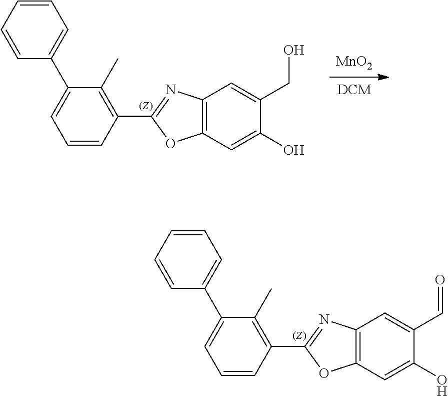

To 5-(hydroxymethyl)-2-(2-methyl-[1,1′-biphenyl]-3-yl)benzo[d]oxazol-6-ol (0.42 g) was added DCM (15 mL), MnO2 (4 g), stirred overnight at room temperature. The reaction solution was filtered with Celite, and the filtrate was concentrated. The crude product was purified by column chromatography (hexane/EA=30/1) to afford 0.1 g 1-(6-hydroxy-2-(2-methyl-[1,1′-biphenyl]-3-yl)benzo[d]oxazol-5-yl)ethan-1-one as a brown solid. LCMS: [M+H]=344.0.

### Step 3: Preparation of 2-(2-methyl-[1,1′-biphenyl]-3-yl)-5-((methylamino)methyl)benzo[d]oxazol-6-ol

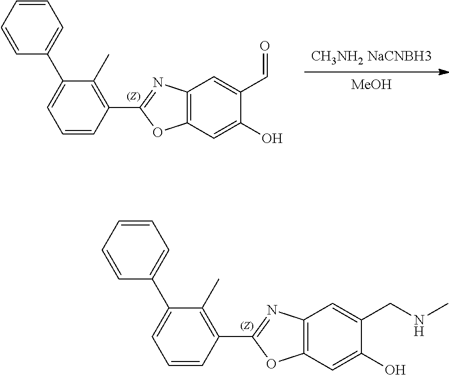 [SMILES: "MeOH" ; Cc1c(-c2ccccc2)cccc1-c1nc2cc(C=O)c(O)cc2o1 ; "CH3NH2 NaCNBH3" ; CNCc1cc2nc(-c3cccc(-c4ccccc4)c3C)oc2cc1O]

To 1-(6-hydroxy-2-(2-methyl-[1,1′-biphenyl]-3-yl)benzo[d]oxazol-5-yl)ethan-1-one (0.1 g) was added MeOH (2 mL), and methylamine in MeOH (5 mL) was added slowly, stirred at room temperature for 2 h, and NaCNBH₃ (0.1 g) was added. Stirred at temperature for 1 h, concentrated, the crude product was purified by column chromatography (DCM/MeOH=30/1) to afford 0.06 g 2-(2-methyl-[1,1′-biphenyl]-3-yl)-5-((methylamino)methyl)benzo[d]oxazol-6-ol as a light yellow solid. LCMS: [M+H]=345.1.

### Step 4: Preparation of 8-methyl-2-(2-methyl-[1,1′-biphenyl]-3-yl)-6,7,8,9-tetrahydrooxazolo[5′,4′:4,5]benzo[1,2-f][1,4]oxazepine

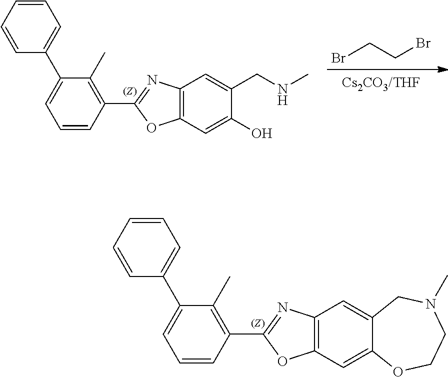

To 2-(2-methyl-[1,1′-biphenyl]-3-yl)-5-((methylamino)methyl)benzo[d]oxazol-6-ol (60 mg) was added CS₂CO₃(100 mg), THF (5 ml), and 1,2-dibromoethane (2 ml), stirred at 80° C. for 3 h, concentrated, the crude product was purified by flash column chromatography (hexane/EA=30/1) to afford 9 mg 8-methyl-2-(2-methyl-[1,1′-biphenyl]-3-yl)-6,7,8,9-tetrahydrooxazolo[5′,4′:4,5]benzo[1,2-f][1,4]oxazepine as a almost white solid. LCMS:[M+H]=371.1

Prepare the following examples (shown in Table 4) essentially as described for Example 67 using the corresponding start materials and intermediates.

TABLE 4

| | | | Physical |
|---|---|---|---|
| EX | | | Data (MS) |
| No. | Chemical Name | Structure | (M + H)⁺ |
| 67 | 8-methyl-2-(2-methyl-[1,1′- biphenyl]-3-yl)-6,7,8,9- tetrahydrooxazolo[5′,4′:4,5]benzo [1,2-f][1,4]oxazepine | 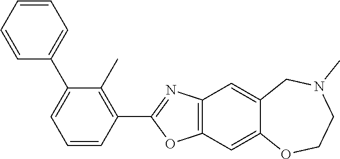 [SMILES: Cc1c(-c2ccccc2)cccc1-c1nc2cc3c(cc2o1)OCCN(C)C3] | 371.2 |
| 68 | 1-((2-(2-methyl-[1,1′-biphenyl]-3- yl)-8-oxo-7,8- dihydrobenzofuro[5,4-d]oxazol-5- yl)methyl)piperidine-2-carboxylic acid | 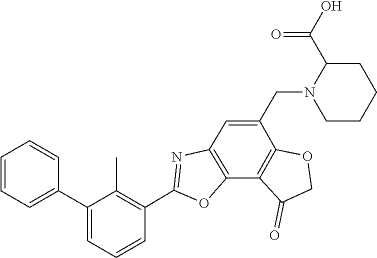 [SMILES: Cc1c(-c2ccccc2)cccc1-c1nc2cc(CN3CCCCC3C(=O)O)c3c(c2o1)C(=O)CO3] | 483.2 |
| 69 | 1-((8-(2-methyl-[1,1′-biphenyl]-3- yl)oxazolo[5,4- c][1,2,4]triazolo[1,5-a]pyridin-5- yl)methyl)piperidine-2-carboxylic acid | 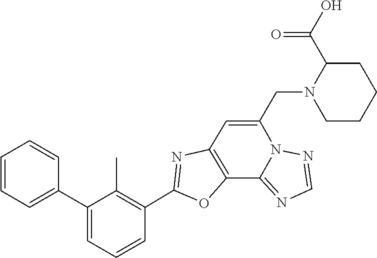 [SMILES: Cc1c(-c2ccccc2)cccc1-c1nc2cc(CN3CCCCC3C(=O)O)n3ncnc3c2o1] | 468.2 |
| 70 | (S)-2-(2-methyl-[1,1′-biphenyl]-3- yl)-6,7,8,9- tetrahydrooxazolo[5′,4′:4,5]benzo [1,2-f][1,4]oxazepine-7-carboxylic acid | 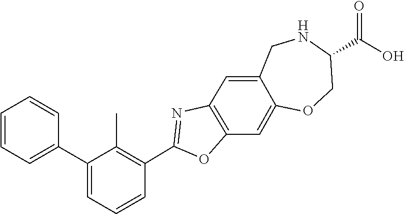 [SMILES: Cc1c(-c2ccccc2)cccc1-c1nc2cc3c(cc2o1)OC[C@@H](C(=O)O)NC3] | 401.2 |
| 71 | 2-(2-(2-methyl-[1,1′-biphenyl]-3- yl)-6,7- dihydrooxazolo[5′,4′:4,5]benzo [1,2-f][1,4]oxazepin-8(9H)-yl)acetic acid | 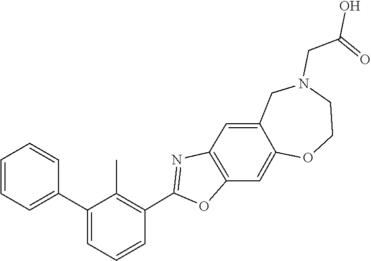 [SMILES: Cc1c(-c2ccccc2)cccc1-c1nc2cc3c(cc2o1)OCCN(CC(=O)O)C3] | 415.2 |
| 72 | (R)-2-(2-methyl-[1,1′-biphenyl]-3- yl)-6,7,8,9- tetrahydrooxazolo[5′,4′:4,5]benzo [1,2-f][1,4]oxazepine-7-carboxylic acid | 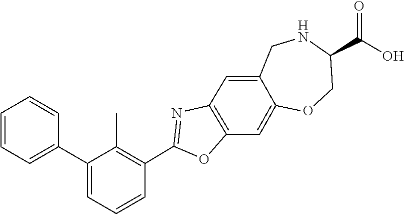 [SMILES: Cc1c(-c2ccccc2)cccc1-c1nc2cc3c(cc2o1)OC[C@H](C(=O)O)NC3] | 401.2 |
| 73 | 1-((2-(2-methyl-[1,1′-biphenyl]-3- yl)-8-oxo-7,8- dihydrobenzofuro[5,4-d]oxazol-5- yl)methyl)piperidine-2-carboxylic acid | 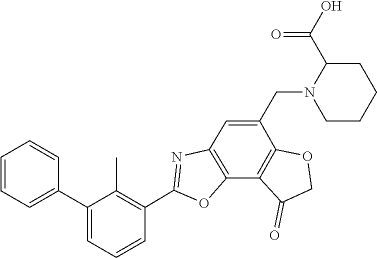 [SMILES: Cc1c(-c2ccccc2)cccc1-c1nc2cc(CN3CCCCC3C(=O)O)c3c(c2o1)C(=O)CO3] | 483.2 |

### Examples for Comparison

Prepare the following comparison examples (as shown in Table 5) essentially as described for Example 26 and Example 31 in WO2017087777.

TABLE 5

| Com. | | | PD-1/PD- |
|---|---|---|---|
| EX. | | | L1 HTRF |
| No. | Chemical Name | Structure | IC50(nM) |
| 1 | 1-((6-(cyanomethoxy)-2- (2-methyl-[1,1′-biphenyl]- 3-yl)benzo[d]oxazol-5- yl)methyl)piperidine-2- carboxylic acid | 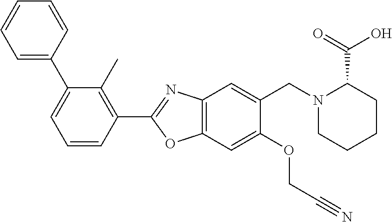 [SMILES: Cc1c(-c2ccccc2)cccc1-c1nc2cc(CN3CCCC[C@H]3C(=O)O)c(OCC#N)cc2o1] | * |
| 2 | 2-(((6-methoxy-2-(2- methyl-[1,1′-biphenyl]-3- yl)benzo[d]oxazol-5- yl)methyl)amino)ethan-1- ol | 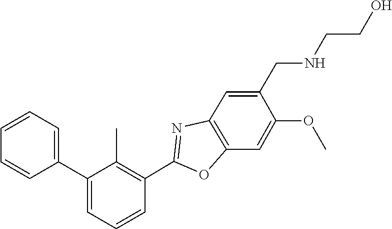 [SMILES: COc1cc2oc(-c3cccc(-c4ccccc4)c3C)nc2cc1CNCCO] | * |

*Date obtain for the above EXAMPLES FOR COMPARISON using the PD-1/PD-L1 homogenous time-resolved fluorescence(HTRF) binding assay described in below.

Resolved Fluorescence (HTRF) Binding Assay

The assays were conducted in a standard black 384-well polystyrene plate with a final volume of 20 μL. Inhibitors were first serially diluted in DMSO and then added to the plate wells before the addition of other reaction components. The final concentration of DMSO in the assay was 1%. The assays were carried out at 25° C. in the PBS buffer (pH 7.4) with 0.05% Tween-20 and 0.1% BSA. Recombinant human PD-L1 protein (19-238) with a His-tag at the C-terminus was purchased from AcroBiosy stems (PD1-H5229). Recombinant human PD-1 protein (25-167) with Fc tag at the C-terminus was also purchased from AcroBiosystems (PD1-H5257). PD-L1 and PD-1 proteins were diluted in the assay buffer and 10 μL was added to the plate well. Plates were centrifuged and proteins were preincubated with inhibitors for 40 min. The incubation was followed by the addition of 10 μL of HTRF detection buffer supplemented with Europium cryptate-labeled anti-human IgG (PerkinElmer-AD0212) specific for Fc and anti-His antibody conjugated to SureLight®-Allophycocyanin (APC, PerkinElmer-AD0059H). After centrifugation, the plate was incubated at 25° C. for 60 min. Before reading on a PHERAstar FS plate reader (665 nm/620 nm ratio). Final concentrations in the assay were −3 nM PD1, 10 nM PD-L1, 1 nM europium anti-human IgG and 20 nM anti-His-Allophycocyanin. IC₅₀ determination was performed by fitting the curve of percent control activity versus the log of the inhibitor concentration using the GraphPad Prism 5.0 software.

Compounds of the present disclosure, as exemplified in the Examples, showed IC₅₀ values in the following ranges: “*” stands for “0.1 nM<IC₅₀ [symbol] 10 nM”; “**” stands for “10 nM<IC₅₀ [symbol] 100 nM”; “***” stands for “100<IC₅₀ [symbol] 1000 nM”.

Data obtained for the Example compounds using the PD-1/PD-L1 homogenous time-resolved fluorescence (HTRF) binding assay described in Example A is provided in Table 6.

TABLE 6

| EX No. | IC₅₀ |
|---|---|
| 1 | 1.9 |
| 2 | * |
| 3 | ** |
| 4 | * |
| 5 | * |
| 6 | ** |
| 7 | * |
| 8 | ** |
| 9 | * |
| 10 | * |
| 11 | ** |
| 12 | * |
| 13 | * |
| 14 | * |
| 15 | ** |
| 16 | * |
| 17 | * |
| 18 | * |
| 19 | * |
| 20 | 1.8 |
| 21 | ** |
| 22 | 1.8 |
| 23 | ** |
| 24 | 7.2 |
| 25 | 7.4 |
| 26 | * |
| 27 | * |
| 28 | 1.4 |
| 29 | * |
| 30 | 4.8 |
| 31 | 3.2 |
| 32 | * |
| 33 | * |
| 34 | ** |
| 35 | * |
| 36 | * |
| 37 | ** |
| 38 | * |
| 39 | 1.1 |
| 40 | * |
| 41 | ** |
| 42 | 3.4 |
| 43 | 1.8 |
| 44 | * |
| 45 | ** |
| 46 | * |
| 47 | * |
| 48 | * |
| 49 | * |
| 50 | * |
| 51 | ** |
| 52 | ** |
| 53 | ** |
| 54 | ** |
| 55 | ** |
| 56 | ** |
| 57 | ** |
| 58 | ** |
| 59 | ** |
| 60 | ** |
| 61 | ** |
| 62 | ** |
| 63 | * |
| 64 | * |
| 65 | 5 |
| 66 | * |
| 67 | *** |
| 68 | *** |
| 69 | *** |
| 70 | *** |
| 71 | *** |
| 72 | *** |
| 73 | *** |

As shown in Table 5 and Table 6, the most exemplified compounds of the present invention display the same level inhibition as the known compounds, Coin. EX. No. 1 and 2 in Table 5.

Pharmacokinetic Assay

Healthy adult male rats were subjected to single-dose of the test compounds with 10% DMSO, 10% Kolliphor® HS 15 and 80% Saline as excipients, and the rats were giving oral administration (intragastric administration) with the compounds at a dose of 25 mg/kg, Before the experiment, the animals were fasted overnight, and the fasting time last from 12 hrs prior to the administration to 4 hrs after the administration. Time of blood collection: 15 min, 30 min, 1 h, 2 h, 4 h, 7 h, 24 h. Approximately 0.3 mL of whole blood was collected from retro-orbital venous sinus, and placed into tubes that contained EDTA as an anticoagulant. The samples were centrifuged at 4° C. and 4000 rpm for 5 min. The plasma was transferred into centrifuge tubes, and stored at −20° C. till being analyzed. Concentrations of test compounds in the plasma samples were analyzed with liquid chromatography-tandem mass spectrometry (LC-MS/MS). Plasma concentration-time data of individual animals was analyzed using WinNonlin (version 4.1; Pharsight) software. Non-compartmental model was introduced in concentration analysis. The pharmacokinetic parameters of the test compounds were calculated. The data is shown in Table 7.

TABLE 7

| | Dose | T_{1/2} | Tₘₐₓ | Cₘₐₓ | AUC |
|---|---|---|---|---|---|
| Examples | (mg/kg) | (hr) | (hr) | (ng/mL) | (hr*ng/mL) |
| Com. EX. No. 1 | 25 | 3.42 | 0.5 | 881 | 5840 |
| Com. EX. No. 2 | 25 | 14.4 | 4.0 | 329 | 5154 |
| Compound 2 | 25 | 17.7 | 1.0 | 12167 | 124960 |
| Compound 20 | 25 | 7.52 | 1.0 | 8270 | 74524 |
| Compound 22 | 25 | 42.3 | 1.0 | 10327 | 151108 |
| Compound 28 | 25 | 41.2 | 0.3 | 14447 | 318799 |
| Compound 29 | 25 | 4.27 | 1.0 | 23300 | 200701 |
| Compound 36 | 25 | 9.3 | 0.5 | 10167 | 118937 |
| Compound 39 | 25 | 10.8 | 1.0 | 30967 | 121277 |
| Compound 52 | 25 | 7.72 | 2.0 | 20660 | 246225 |

As shown in Table 7, we can see, the exemplified compounds of the present invention display unexpectedly better pharmacokinetic properties than the known compounds, Com. EX. No. 1 and 2 in Table 5.

1. A compound of Formula I, or a stereoisomer, tautomer, pharmaceutically acceptable salt, prodrug, chelate, non-covalent complex, or solvate thereof, 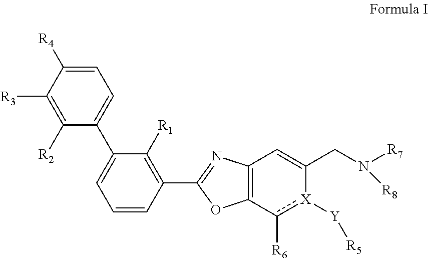
wherein,
X is selected from C, or N; [symbol]
is a single bond or a double bond;
R₁ is selected from H, halogen, CN, —C₁₋₈alkyl, —C₁₋₄haloalkyl, or —OC₁₋₈ alkyl;
R₂, R₃ and R₄ are each independently selected from H, —OH, halogen, —CN, —C₁₋₈alkyl, —C₂₋₈alkenyl, —C₁₋₈alkoxyl, —O—C₁₋₄alkyl-C₅₋₁₀heterocycle, —C₃₋₁₀heteroaryl, —NHCO—C₈₋₁₀heteroaryl; —NHCO—C₁₋₄alkyl-C₅₋₁₀heterocycle; wherein —C₁₋₈alkyl, —C₁₋₈alkoxyl, —O—C₁₋₄alkyl-C₅₋₁₀heterocycle, —C₃₋₁₀heteroaryl, —NHCO—C₈₋₁₀heteroaryl, —NHCO—C₁₋₄alkyl-C₅₋₁₀heterocycle substituted with —C₁₋₈alkyl, —C₁₋₈alkoxyl, —C₃₋₁₀cycloalkyl, —C₃₋₁₀cycloalkyl-O—C₁₋₈alkyl; or
R₃ and R₄ together with the atoms to which they are attached form a 5- to 6-member heterocyclic ring; wherein the heterocyclic ring optionally comprising 1, 2 or 3 hetero atoms independently selected from N, S, or O;
Y is selected from absent, O, S, —NR₉—;
R₉ is selected from H, —C₁₋₈alkyl, or —C₁₋₈ haloalkyl;
R₅ is selected from H, halogen, CN, —C₁₋₈alkyl, —C₁₋₄haloalkyl, —C₂₋₈alkenyl, sulfonyl, sulfinyl, provided that if Y is O, then R₅ is not —C₁₋₈alkyl;
R₆ is H, or R₅ and R₆ together with the atoms to which they are attached form a 5- to 6-member heterocyclic ring; wherein the heterocyclic ring optionally comprising 1, 2 or 3 hetero atoms independently selected from N, S, or O; the heterocyclic ring optionally substituted with oxo, —C₁₋₈ alkyl, —C₀₋₄ alkyl-COOH, —C₀₋₄ alkyl-OH;
R₇ and R₈ are each independently selected from H, —C₁₋₈ alkyl, —C₁₋₆ alkyl-COOH, —C₅₋₆ aryl, wherein —C₁₋₆ alkyl-COOH and —C₅₋₆ aryl optionally substituted with —C₁₋₈ alkyl, —C₀₋₄ alkyl-COOH, —C₀₋₄ alkyl-OH; or
R₇ and R₈ together with the atoms to which they are attached form a 3- to 7-member heterocyclic ring; wherein the heterocyclic ring optionally comprising 1, 2 or 3 hetero atoms independently selected from N, S, or O; the heterocyclic ring optionally substituted with oxo, —C₁₋₈ alkyl, —C₀₋₄ alkyl-COOH, —C₀₋₄ alkyl-OH;
R₅ and R₈ together with the atoms to which they are attached form a 6- to 10-member heterocyclic ring; wherein the heterocyclic ring optionally comprising 1, 2 or 3 hetero atoms independently selected from N, S, or O; the heterocyclic ring optionally substituted with oxo, —C₁₋₈ alkyl, —C₀₋₄ alkyl-COOH, —C₀₋₄ alkyl-OH.

2. The compound of claim 1, wherein Y is selected from absent, —O—, —S—, —NH—.

3. The compound of claim 1, wherein R₁ is selected from CH₃ and CN.

4. The compound of claim 1, wherein R₅ is selected from H, —CH₃, —CHF₂, —CF₃, —CH₂CF₃, F, Cl, CN, 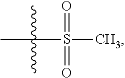 [SMILES: *S(C)(=O)=O]
and provided that if Y is O, then R₅ is not —CH₃.

5. The compound of Formula II, or a stereoisomer, tautomer, pharmaceutically acceptable salt, prodrug, chelate, non-covalent complex, or solvate thereof, 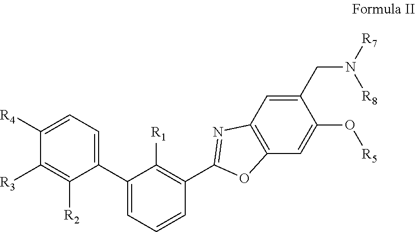
wherein,
R₁ is selected from H, halogen, CN, —C₁₋₈alkyl, —C₁₋₄haloalkyl, or —OC₁₋₈ alkyl;
R₂, R₃ and R₄ are each independently selected from H, halogen, CN, —C₁₋₈alkyl, or —C₁₋₄haloalkyl;
R₅ is selected from H, —C₁₋₄haloalkyl, —SO₂—C₁₋₄ alkyl;
R₇ and R₈ are each independently selected from H, —C₁₋₈ alkyl, —C₁₋₆ alkyl-COOH, —C₅₋₆ aryl, wherein —C₁₋₆ alkyl-COOH and —C₅₋₆ aryl optionally substituted with —C₁₋₈ alkyl, —C₀₋₄ alkyl-COOH, —C₀₋₄ alkyl-OH; or
R₇ and R₈ together with the atoms to which they are attached form a 3- to 7-member heterocyclic ring; wherein the heterocyclic ring optionally comprising 1, 2 or 3 hetero atoms independently selected from N, S, or O; the heterocyclic ring optionally substituted with oxo, —C₁₋₈ alkyl, —C₀₋₄ alkyl-COOH, —C₀₋₄ alkyl-OH.

6. The compound of claim 5, wherein R₁ is selected from CH₃ and CN.

7. The compound of claim 5, wherein R₂, R₃ and R₄ are each independently selected from H, CN, or F.

8. The compound of claim 5, wherein R₅ is —C₁₋₄haloalkyl.

9. The compound of claim 8, wherein R₅ is —C₁₋₄alkyl substituted by 1-3 fluorine atoms.

10. The compound of claim 9, wherein R₅ is methyl or ethyl substituted by 2-3 fluorine atoms.

11. The compound of claim 10, wherein R₅ is —CF₃, —CHF₂, —CH₂CHF₂, or —CH₂CF₃.

12. The compound of claim 5, wherein R₅ is —SO₂—C₁₋₄ alkyl.

13. The compound of claim 12, wherein R₅ is —SO₂—CH₃.

14. The compound of claim 5, wherein R₇ and R₈ are each independently selected from H, CH₃,

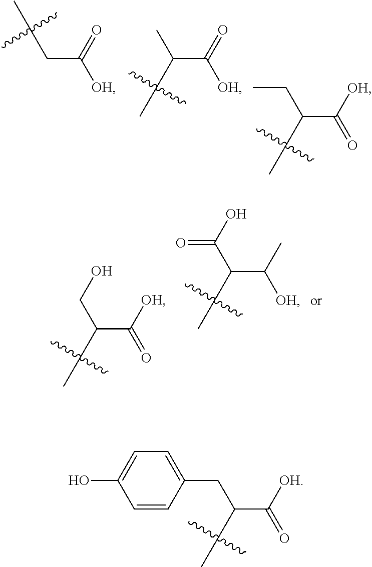

15. The compound of claim 5, wherein R₇ and R₈ together with the atoms to which they are attached form a 4- to 6-member heterocyclic ring; wherein the heterocyclic ring substituted with —C₁₋₄ alkyl, —C₀₋₄ alkyl-COOH, or —C₀₋₄ alkyl-OH.

16. The compound of claim 15, wherein heterocyclic ring is

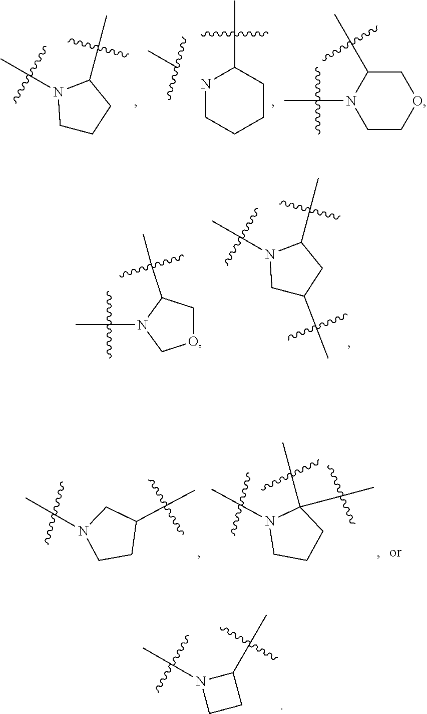

17. The compound of claim 15, wherein the substituted heterocyclic ring is selected from

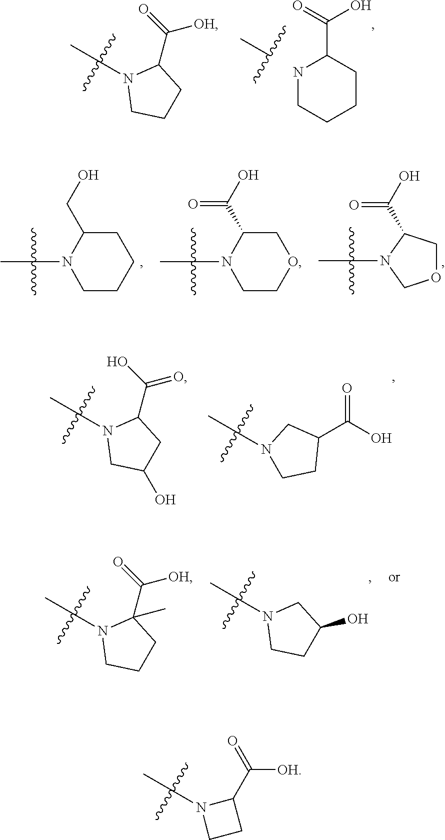

18. A compound of Formula III, or a stereoisomer,
tautomer, pharmaceutically acceptable salt, prodrug, chelate, non-covalent complex, or solvate thereof, 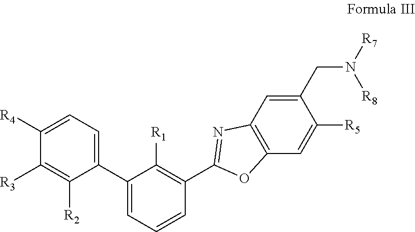
wherein,
R₁ is selected from H, halogen, CN, —C₁₋₈alkyl, —C₁₋₄haloalkyl, or —OC₁₋₈ alkyl;
R₂, R₃ and R₄ are each independently selected from H, halogen, CN, —C₁₋₈alkyl, or —C₁₋₄haloalkyl;
R₅ is selected from H, halogen, CN, —C₁₋₈alkyl, —C₁₋₄haloalkyl, —NH—C₁₋₄ alkyl, —S—C₁₋₄ alkyl, sulfonyl, or sulfinyl;
R₇ and R₈ are each independently selected from H, —C₁₋₈ alkyl, —C₁₋₆ alkyl-COOH, —C₅₋₆ aryl, wherein —C₁₋₆ alkyl-COOH and —C₅₋₆ aryl optionally substituted with —C₁₋₈ alkyl, —C₀₋₄ alkyl-COOH, —C₀₋₄ alkyl-OH; or
R₇ and R₈ together with the atoms to which they are attached form a 3- to 7-member heterocyclic ring; wherein the heterocyclic ring optionally comprising 1, 2 or 3 hetero atoms independently selected from N, S, or O; the heterocyclic ring optionally substituted with oxo, —C₁₋₈ alkyl, —C₀₋₄ alkyl-COOH, —C₀₋₄ alkyl-OH.

19. The compound of claim 18, R₁ is selected from CH₃, or CN.

20. The compound of claim 18, wherein R₂, R₃ and R₄ are each independently selected from H, CN, or F.

21. The compound of claim 18, wherein R₅ is selected from F, Cl, —CH₃, —CF₃, —S—CH₃, —SO—CH₃, —SO₂—CH₃, —CN, or —NHCH₃.

22. The compound of claim 18, wherein R₇ and R₈ are each independently selected from H, CH₃,

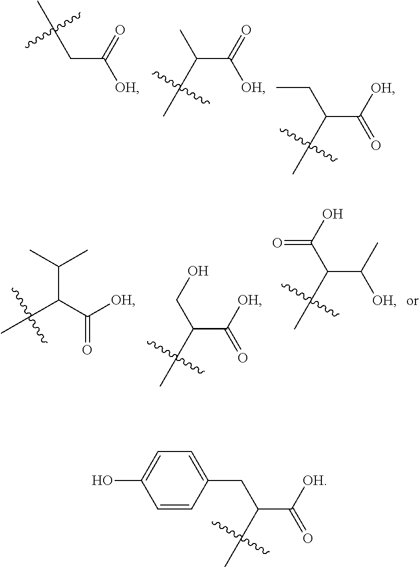

23. The compound of claim 18, wherein R₇ and R₈ together with the atoms to which they are attached form a 5- to 6-member heterocyclic ring; wherein the heterocyclic ring substituted with —C₀₋₄ alkyl-COOH.

24. The compound of claim 23, wherein the substituted heterocyclic ring is selected from

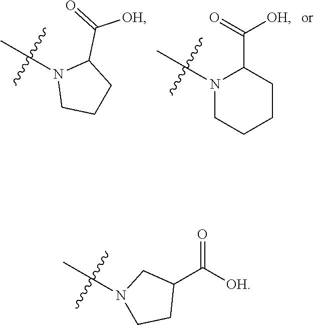 [SMILES: *N1CCCC1C(=O)O ; *N1CCCCC1C(=O)O ; *N1CCC(C(=O)O)C1]

25. The compound of Formula I according to claim 1, wherein the compound is:
1) (S)-1-((2-(2-methyl-[1,1′-biphenyl]-3-yl)-6-(methylthio)benzo[d]oxazol-5-yl)methyl)piperidine-2-carboxylic acid;
2) ((2-(2-methyl-[1,1′-biphenyl]-3-yl)-6-(methylthio)benzo[d]oxazol-5-yl)methyl)-L-proline;
3) ((2-(2-methyl-[1,1′-biphenyl]-3-yl)-6-(methylthio)benzo[d]oxazol-5-yl)methyl)-D-allothreonine;
4) ((2-(2-methyl-[1,1′-biphenyl]-3-yl)-6-(methylthio)benzo[d]oxazol-5-yl)methyl)-L-allothreonine;
5) ((2-(2-methyl-[1,1′-biphenyl]-3-yl)-6-(methylthio)benzo[d]oxazol-5-yl)methyl)-L-alanine;
6) ((2-(2-methyl-[1,1′-biphenyl]-3-yl)-6-(methylthio)benzo[d]oxazol-5-yl)methyl)-D-alanine;
7) ((2-(2-methyl-[1,1′-biphenyl]-3-yl)-6-(methylthio)benzo[d]oxazol-5-yl)methyl)-D-proline;
8) (1-((2-(2-methyl-[1,1′-biphenyl]-3-yl)-6-(methylthio)benzo[d]oxazol-5-yl)methyl)piperidin-2-yl)methanol;
9) (S)-4-((2-(2-methyl-[1,1′-biphenyl]-3-yl)-6-(methylthio)benzo[d]oxazol-5-yl)methyl)morpholine-3-carboxylic acid;
10) (S)-2-(((2-(2-methyl-[1,1′-biphenyl]-3-yl)-6-(methylthio)benzo[d]oxazol-5-yl)methyl)amino)butanoic acid;
11) (R)-2-(((2-(2-methyl-[1,1′-biphenyl]-3-yl)-6-(methylthio)benzo[d]oxazol-5-yl)methyl)amino)butanoic acid;
12) ((2-(2-methyl-[1,1′-biphenyl]-3-yl)-6-(methylthio)benzo[d]oxazol-5-yl)methyl)-D-serine;
13) ((2-(2-methyl-[1,1′-biphenyl]-3-yl)-6-(methylthio)benzo[d]oxazol-5-yl)methyl)-L-serine;
14) ((2-(2-cyano-[1,1′-biphenyl]-3-yl)-6-(methylthio)benzo[d]oxazol-5-yl)methyl)-L-proline;
15) ((2-(2,2′-dicyano-[1,1′-biphenyl]-3-yl)-6-(methylthio)benzo[d]oxazol-5-yl)methyl)-L-proline;
16) 2-methyl-1-((2-(2-methyl-[1,1′-biphenyl]-3-yl)-6-(methylthio)benzo[d]oxazol-5-yl)methyl)pyrrolidine-2-carboxylic acid;
17) ((2-(2-cyano-[1,1′-biphenyl]-3-yl)-6-methylbenzo[d]oxazol-5-yl)methyl)-D-proline;
18) ((2-(2-cyano-[1,1′-biphenyl]-3-yl)-6-methylbenzo[d]oxazol-5-yl)methyl)-L-proline;
19) (S)-4-((2-(2-cyano-[1,1′-biphenyl]-3-yl)-6-methylbenzo[d]oxazol-5-yl)methyl)morpholine-3-carboxylic acid;
20) ((6-chloro-2-(2-methyl-[1,1′-biphenyl]-3-yl)benzo[d]oxazol-5-yl)methyl)-L-proline;
21) ((6-hydroxy-2-(2-methyl-[1,1′-biphenyl]-3-yl)benzo[d]oxazol-5-yl)methyl)-L-proline;
22) (S)-1-((6-methyl-2-(2-methyl-[1,1′-biphenyl]-3-yl)benzo[d]oxazol-5-yl)methyl)piperidine-2-carboxylic acid;
23) (S)-1-((6-fluoro-2-(2-methyl-[1,1′-biphenyl]-3-yl)benzo[d]oxazol-5-yl)methyl)pyrrolidin-3-ol;
24) ((6-fluoro-2-(2-methyl-[1,1′-biphenyl]-3-yl)benzo[d]oxazol-5-yl)methyl)-L-proline;
25) (S)-1-((6-fluoro-2-(2-methyl-[1,1′-biphenyl]-3-yl)benzo[d]oxazol-5-yl)methyl)piperidine-2-carboxylic acid;
26) 1-((6-cyano-2-(2-methyl-[1,1′-biphenyl]-3-yl)benzo[d]oxazol-5-yl)methyl)piperidine-2-carboxylic acid;
27) 1-((2-(2-methyl-[1,1′-biphenyl]-3-yl)-6-(methylamino)benzo[d]oxazol-5-yl)methyl)piperidine-2-carboxylic acid;
28) ((2-(2-methyl-[1,1′-biphenyl]-3-yl)-6-(trifluoromethyl)benzo[d]oxazol-5-yl)methyl)-L-proline;
29) (S)-1-((6-(difluoromethoxy)-2-(2-methyl-[1,1′-biphenyl]-3-yl)benzo[d]oxazol-5-yl)methyl)piperidine-2-carboxylic acid;
30) ((6-(difluoromethoxy)-2-(2-methyl-[1,1′-biphenyl]-3-yl)benzo[d]oxazol-5-yl)methyl)-D-allothreonine;
31) ((6-(difluoromethoxy)-2-(2-methyl-[1,1′-biphenyl]-3-yl)benzo[d]oxazol-5-yl)methyl)-L-allothreonine;
32) ((6-(difluoromethoxy)-2-(2-methyl-[1,1′-biphenyl]-3-yl)benzo[d]oxazol-5-yl)methyl)-L-tyrosine hydrochloride;
33) ((6-(difluoromethoxy)-2-(2-methyl-[1,1′-biphenyl]-3-yl)benzo[d]oxazol-5-yl)methyl)-L-alanine;
34) ((6-(difluoromethoxy)-2-(2-methyl-[1,1′-biphenyl]-3-yl)benzo[d]oxazol-5-yl)methyl)-D-alanine;
35) ((6-(difluoromethoxy)-2-(2-methyl-[1,1′-biphenyl]-3-yl)benzo[d]oxazol-5-yl)methyl)glycine;
36) ((6-(difluoromethoxy)-2-(2-methyl-[1,1′-biphenyl]-3-yl)benzo[d]oxazol-5-yl)methyl)-L-proline;
37) ((6-(difluoromethoxy)-2-(2-methyl-[1,1′-biphenyl]-3-yl)benzo[d]oxazol-5-yl)methyl)-D-proline;
38) (1-((6-(difluoromethoxy)-2-(2-methyl-[1,1′-biphenyl]-3-yl)benzo[d]oxazol-5-yl)methyl)piperidin-2-yl)methanol;
39) (S)-4-((6-(difluoromethoxy)-2-(2-methyl-[1,1′-biphenyl]-3-yl)benzo[d]oxazol-5-yl)methyl)morpholine-3-carboxylic acid;
40) (S)-2-(((6-(difluoromethoxy)-2-(2-methyl-[1,1′-biphenyl]-3-yl)benzo[d]oxazol-5-yl)methyl)amino)butanoic acid;
41) (R)-2-(((6-(difluoromethoxy)-2-(2-methyl-[1,1′-biphenyl]-3-yl)benzo[d]oxazol-5-yl)methyl)amino)butanoic acid;
42) ((6-(difluoromethoxy)-2-(2-methyl-[1,1′-biphenyl]-3-yl)benzo[d]oxazol-5-yl)methyl)-D-serine;
43) ((6-(difluoromethoxy)-2-(2-methyl-[1,1′-biphenyl]-3-yl)benzo[d]oxazol-5-yl)methyl)-L-serine;
44) ((2-(2-cyano-[1,1′-biphenyl]-3-yl)-6-(difluoromethoxy)benzo[d]oxazol-5-yl)methyl)-L-proline;
45) ((2-(2,2′-dicyano-[1,1′-biphenyl]-3-yl)-6-(difluoromethoxy)benzo[d]oxazol-5-yl)methyl)-L-proline;
46) 1-((6-(difluoromethoxy)-2-(2-methyl-[1,1′-biphenyl]-3-yl)benzo[d]oxazol-5-yl)methyl)-2-methylpyrrolidine-2-carboxylic acid;
47) ((6-(difluoromethoxy)-2-(3-(2,3-dihydrobenzo[b][1,4]dioxin-6-yl)-2-methylphenyl)benzo[d]oxazol-5-yl)methyl)-L-proline;
48) 1-((6-(difluoromethoxy)-2-(2-methyl-[1,1′-biphenyl]-3-yl)benzo[d]oxazol-5-yl)methyl)azetidine-2-carboxylic acid;
49) ((6-(difluoromethoxy)-2-(3-(2,3-dihydrobenzo[b][1,4]dioxin-6-yl)-2-methylphenyl)benzo[d]oxazol-5-yl)methyl)-L-proline;
50) 1-((6-(difluoromethoxy)-2-(2-methyl-[1,1′-biphenyl]-3-yl)benzo[d]oxazol-5-yl)methyl)-2-methylpyrrolidine-2-carboxylic acid;
51) ((2-(2′-cyano-2-methyl-[1,1′-biphenyl]-3-yl)-6-(difluoromethoxy)benzo[d]oxazol-5-yl)methyl)proline;
52) ((6-(difluoromethoxy)-2-(2′-fluoro-2-methyl-[1,1′-biphenyl]-3-yl)benzo[d]oxazol-5-yl)methyl)-L-proline;
53) (S)-3-((6-(difluoromethoxy)-2-(2-methyl-[1,1′-biphenyl]-3-yl)benzo[d]oxazol-5-yl)methyl)oxazolidine-4-carboxylic acid;
54) (S)-1-((2-(2-methyl-[1,1′-biphenyl]-3-yl)-6-(2,2,2-trifluoroethoxy)benzo[d]oxazol-5-yl)methyl)piperidine-2-carboxylic acid;
55) 1-((6-(2,2-difluoroethoxy)-2-(2-methyl-[1,1′-biphenyl]-3-yl)benzo[d]oxazol-5-yl)methyl)piperidine-2-carboxylic acid;
56) ((2-(2-methyl-[1,1′-biphenyl]-3-yl)-6-(2,2,2-trifluoroethoxy)benzo[d]oxazol-5-yl)methyl)-D-alanine;
57) (S)-4-((2-(2-cyano-[1,1′-biphenyl]-3-yl)-6-(2,2,2-trifluoroethoxy)benzo[d]oxazol-5-yl)methyl)morpholine-3-carboxylic acid;
58) (S)-4-((2-(2-methyl-[1,1′-biphenyl]-3-yl)-6-(2,2,2-trifluoroethoxy)benzo[d]oxazol-5-yl)methyl)morpholine-3-carboxylic acid;
59) ((2-(2-methyl-[1,1′-biphenyl]-3-yl)-6-(2,2,2-trifluoroethoxy)benzo[d]oxazol-5-yl)methyl)-L-alanine;
60) ((2-(2-methyl-[1,1′-biphenyl]-3-yl)-6-(2,2,2-trifluoroethoxy)benzo[d]oxazol-5-yl)methyl)-L-proline;
61) 2-methyl-1-((2-(2-methyl-[1,1′-biphenyl]-3-yl)-6-(2,2,2-trifluoroethoxy)benzo[d]oxazol-5-yl)methyl)pyrrolidine-2-carboxylic acid;
62) ((2-(2-methyl-[1,1′-biphenyl]-3-yl)-6-(2,2,2-trifluoroethoxy)benzo[d]oxazol-5-yl)methyl)-D-alanine;
63) ((2-(2-methyl-[1,1′-biphenyl]-3-yl)-6-(trifluoromethoxy)benzo[d]oxazol-5-yl)methyl)-L-proline;
64) ((2-(2-methyl-[1,1′-biphenyl]-3-yl)-6-((methylsulfonyl)oxy)benzo[d]oxazol-5-yl)methyl)-L-proline;
65) ((2-(2-methyl-[1,1′-biphenyl]-3-yl)-6-(methylsulfinyl)benzo[d]oxazol-5-yl)methyl)-L-proline;
66) ((2-(2-methyl-[1,1′-biphenyl]-3-yl)-6-(methylsulfonyl)benzo[d]oxazol-5-yl)methyl)-L-proline;
67) 8-methyl-2-(2-methyl-[1,1′-biphenyl]-3-yl)-6,7,8,9-tetrahydrooxazolo[5′,4′:4,5]benzo[1,2-f][1,4]oxazepine;
68) 1-((2-(2-methyl-[1,1′-biphenyl]-3-yl)-8-oxo-7,8-dihydrobenzofuro[5,4-d]oxazol-5-yl)methyl)piperidine-2-carboxylic acid;
69) 1-((8-(2-methyl-[1,1′-biphenyl]-3-yl)oxazolo[5,4-c][1,2,4]triazolo[1,5-a]pyridin-5-yl)methyl)piperidine-2-carboxylic acid;
70) (S)-2-(2-methyl-[1,1′-biphenyl]-3-yl)-6,7,8,9-tetrahydrooxazolo[5′,4′:4,5]benzo[1,2-f][1,4]oxazepine-7-carboxylic acid;
71) 2-(2-(2-methyl-[1,1′-biphenyl]-3-yl)-6,7-dihydrooxazolo[5′,4′:4,5]benzo[1,2-f][1,4]oxazepin-8(9H)-yl)acetic acid;
72) (R)-2-(2-methyl-[1,1′-biphenyl]-3-yl)-6,7,8,9-tetrahydrooxazolo[5′,4′:4,5]benzo[1,2-f][1,4]oxazepine-7-carboxylic acid;
73) 1-((2-(2-methyl-[1,1′-biphenyl]-3-yl)-8-oxo-7,8-dihydrobenzofuro[5,4-d]oxazol-5-yl)methyl)piperidine-2-carboxylic acid.

26. A pharmaceutical composition comprising a compound of claim 1, or a pharmaceutically acceptable salt or a stereoisomer thereof, and at least one pharmaceutically acceptable carrier or excipient.

27. A method of inhibiting PD-1/PD-L1 interaction, said method comprising administering to a patient a compound of claim 1, or a pharmaceutically acceptable salt or a stereoisomer thereof.

28. A method of treating a disease associated with inhibition of PD-1/PD-L1 interaction, said method comprising administering to a patient in need thereof a therapeutically effective amount of a compound of claim 1, or a pharmaceutically acceptable salt or a stereoisomer thereof.

29. The method of claim 28, wherein the disease is colon cancer, gastric cancer, thyroid cancer, lung cancer, leukemia, pancreatic cancer, melanoma, multiple melanoma, brain cancer, renal cancer, prostate cancer, ovarian cancer or breast cancer.

30. A method of enhancing, stimulating and/or increasing the immune response in a patient, said method comprising administering to the patient in need thereof a therapeutically effective amount of a compound of claim 1, or a pharmaceutically acceptable salt or a stereoisomer thereof.

31-34. (canceled)
